(19) United States
(12) Patent Application Publication
(10) Pub. No.: US 2022/0181023 A1
(43) Pub. Date: Jun. 9, 2022

(54) DIGITAL SOLUTIONS FOR DIFFERENTIATING ASTHMA FROM COPD

(71) Applicant: Novartis AG, Basel (CH)

(72) Inventors: Paul Mastoridis, Glassboro, NJ (US); Eli Goldberg, Norfolk, MA (US); Nicholas Vincent Iannotti, Cambridge, MA (US); Hui CAO, Princeton, NJ (US); Pascal Pfister, Mulhouse (FR); Eric Hwai-Yu Yang, Cambridge, MA (US)

(21) Appl. No.: 17/437,336

(22) PCT Filed: Mar. 10, 2020
(86) PCT No.: PCT/IB2020/052063

§ 371 Date: Sep. 8, 2021

Related U.S. Application Data
(60) Provisional application No. 62/817,210, filed on Mar. 12, 2019.

(51) Int. Cl. G16H 50/20; G16H 50/70

(52) U.S. Cl. CPC G16H 50/20; G16H 50/70

(57) ABSTRACT

The present disclosure relates generally to systems and processes for assessing and differentiating asthma and chronic obstructive pulmonary disease (COPD) in a patient, and more specifically to computer-based systems and processes for providing a predicted diagnosis of asthma and/or COPD. In accordance with one or more examples, a computing system receives a set of patient data corresponding to a first patient and determines whether the set of patient data satisfies a set of one or more data-correlation criteria. If the set of one or more data-correlation criteria are satisfied, the computing system applies a first diagnostic model to the set of patient data and determines a first predicted diagnosis of asthma and/or COPD. If the set of one or more data-correlation criteria are not satisfied, the computing system applies a second diagnostic model to the set of patient data and determines a second predicted diagnosis of asthma and/or COPD.

### FIELD

The present disclosure relates generally to systems and processes for assessing and differentiating asthma and chronic obstructive pulmonary disease (COPD) in a patient, and more specifically to computer-based systems and processes for providing a predicted diagnosis of asthma and/or COPD.

### BACKGROUND

Asthma and chronic obstructive pulmonary disease (COPD) are both common obstructive lung diseases affecting millions of individuals around the world. Asthma is a chronic inflammatory disease of hyper-reactive airways, in which episodes are often associated with specific triggers, such as allergens. In contrast, COPD is a progressive disease characterized by persistent airflow limitation due to chronic inflammatory response of the lungs to noxious particles or gases, commonly caused by cigarette smoking.

Despite sharing some key symptoms, such as shortness of breath and wheezing, asthma and COPD are quite different in terms of how they are treated and managed. Drugs for treating asthma and COPD can come from the same class and many of them can be used for both diseases. However, the pathways of treatment and combinations of drugs often differ, especially in different stages of the diseases. Further, while individuals with asthma and COPD are encouraged to avoid their personal triggers, such as pets, tree pollen, and cigarette smoking, some individuals with COPD may also be prescribed oxygen or undergo pulmonary rehabilitation, a program that focuses on learning new breathing strategies, different ways to do daily tasks, and personal exercise training. As such, accurate differentiation of asthma from COPD directly contributes to the proper treatment of individuals with either disease and thus the reduction of exacerbations and hospitalizations.

In order to differentiate between asthma and COPD in patients, physicians typically gather information regarding the patient's symptoms, medical history, and environment. After gathering patient information and data using available processes and tools, the differential diagnosis between asthma and COPD ultimately falls on the physician and thus can be affected by the physician's experience or knowledge. Further, in cases where an individual has long-term asthma or when the onset of asthma occurs later in an individual's life, differentiation between asthma and COPD becomes much more difficult—even with available information and data—due to the similarity of asthma and COPD case histories and symptoms. As a result, physicians often misdiagnose asthma and COPD, resulting in improper therapy, increased morbidity, and decrease of patient quality of life.

Accordingly, there is a need for a more reliable, accurate, and reproducible system and process for differentiating asthma from COPD in patients that does not rely primarily on the experience or knowledge available to physicians.

### SUMMARY

Systems and processes for the diagnostic application of one or more diagnostic models for differentiating asthma from chronic obstructive pulmonary disease (COPD) and providing a predicted diagnosis of asthma and/or COPD are provided. In accordance with one or more examples, a computing device comprises one or more processors, one or more input elements, memory, and one or more programs stored in the memory. The one or more programs include instructions for receiving, via the one or more input elements, a set of patient data corresponding to a first patient, the set of patient data including at least one physiological input based on results of at least one physiological test administered to the first patient. The one or more programs further include instructions for determining, based on the set of patient data, whether a set of one or more data-correlation criteria are satisfied, wherein the set of one or more data-correlation criteria are based on an application of an unsupervised machine learning algorithm to a first historical set of patient data that includes data from a first plurality of patients having one or more phenotypic differences, the phenotypic differences including at least data regarding one or more respiratory conditions. The one or more programs further include instructions for determining, in accordance with a determination that the set of one or more data-correlation criteria are satisfied, a first indication of whether the first patient has one or more respiratory conditions selected from a group consisting of asthma and chronic obstructive pulmonary disease (COPD) based on an application of a first diagnostic model to the set of patient data, wherein the first diagnostic model is based on an application of a first supervised machine learning algorithm to a second historical set of patient data that includes data from a second plurality of patients having one or more phenotypic differences, the phenotypic differences including at least data regarding one or more respiratory conditions. The one or more programs further include instructions for outputting the first indication.

The one or more programs further include instructions for determining, in accordance with a determination that the set of one or more data-correlation criteria are not satisfied, determining a second indication of whether the first patient has one or more respiratory conditions selected from a group consisting of asthma and chronic obstructive pulmonary disease (COPD) based on an application of a second diagnostic model to the set of patient data, wherein the second diagnostic model is based on an application of a second supervised machine learning algorithm to a third historical set of patient data that includes data from a third plurality of patients having one or more phenotypic differences, the phenotypic differences including at least data regarding one or more respiratory conditions, and wherein the third historical set of patient data is different from the second historical set of patient data. The one or more programs further include instructions for outputting the second indication.

The executable instructions for performing the above functions are, optionally, included in a non-transitory computer-readable storage medium or other computer program product configured for execution by one or more processors.

### BRIEF DESCRIPTION OF THE DRAWINGS

FIG. 1 illustrates an exemplary system for differentially diagnosing asthma and COPD in a patient.

FIG. 2 illustrates an exemplary machine learning system in accordance with some embodiments.

FIG. 3 illustrates an exemplary electronic device in accordance with some embodiments.

FIG. 4 illustrates an exemplary, computerized process for generating two supervised machine learning models for differentially diagnosing asthma and COPD in a patient.

FIG. 5 illustrates a portion of an exemplary data set including anonymized electronic health records for a plurality of patients diagnosed with asthma and/or COPD.

FIG. 6 illustrates a portion of an exemplary data set after pre-processing.

FIG. 7 illustrates a portion of an exemplary data set after feature engineering.

FIG. 8 illustrates a portion of an exemplary data set after the application of two unsupervised machine learning algorithms to the exemplary data set and the removal of all outliers/phenotypic misses from the exemplary data set.

FIG. 9 illustrates an exemplary, computerized process for generating a first diagnostic model and a second diagnostic model for differentially diagnosing asthma and COPD in a patient.

FIG. 10 illustrates an exemplary, computerized process for differentially diagnosing asthma and COPD in a patient.

FIG. 11A illustrates two exemplary sets of patient data corresponding to a first patient and a second patient.

FIG. 11B illustrates two exemplary sets of patient data corresponding to a first patient and a second patient after pre-processing.

FIG. 11C illustrates two exemplary sets of patient data after feature engineering.

FIG. 11D illustrates two exemplary sets of patient data after the application of two unsupervised machine learning models to the two exemplary sets of patient data.

FIG. 11E illustrates two exemplary sets of patient data after the application of a separate supervised machine learning model to each of the two exemplary sets of patient data.

FIG. 12 illustrates an exemplary, computerized process for determining a first indication and a second indication of whether a first patient has one or more respiratory conditions selected from a group consisting of asthma and COPD.

FIGS. 13A-H illustrate bar graphs representing exemplary inlier and outlier classification results based on the application of Gaussian mixture models to subsets of a feature-engineered test set of patient data stratified based on gender.

FIG. 14 illustrates a receiver operating characteristic curve representing asthma and/or COPD classification results from the application of a supervised machine learning model (trained using an inlier data set of patients) to a test set of patient data.

### DETAILED DESCRIPTION

The following description sets forth exemplary systems, devices, methods, parameters, and the like. It should be recognized, however, that such description is not intended as a limitation on the scope of the present disclosure but is instead provided as a description of exemplary embodiments. For example, reference is made to the accompanying drawings in which it is shown, by way of illustration, specific example embodiments. It is to be understood that changes can be made to such example embodiments without departing from the scope of the present disclosure.

### 1. Computing System

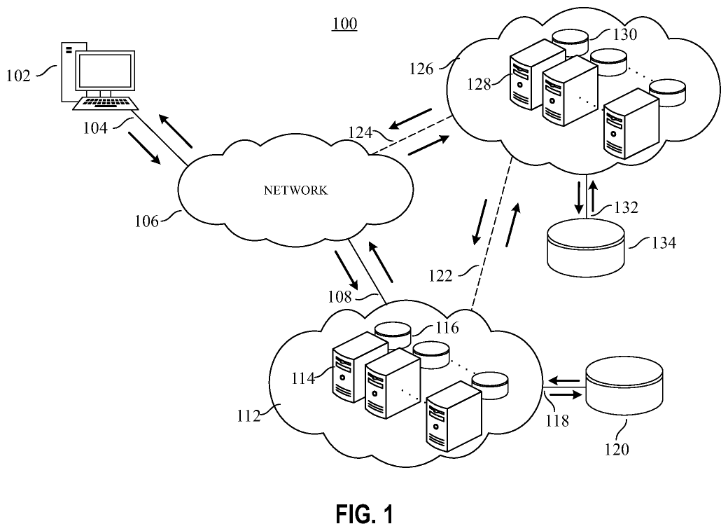

Attention is now directed to examples of electronic devices and systems for performing the techniques described herein in accordance with some embodiments. FIG. 1 illustrates an exemplary system 100 of electronic devices (e.g., such as electronic device 300). System 100 includes a client system 102. In some examples, client system 102 includes one or more electronic devices (e.g., 300). For example, client system 102 can represent a health care provider's (HCP) computing system (e.g., one or more personal computers (e.g., desktop, laptop)) and can be used for the input, collection, and/or processing of patient data by a HCP, as well as for the output of patient data analysis (e.g., prognosis information). For further example, client system 102 can represent a patient's device (e.g., a home-use medical device; a personal electronic device such as a smartphone, tablet, desktop computer, or laptop computer) that is connected to one or more HCP electronic devices and/or to system 108, and that is used for the input and collection of patient data. In some examples, client system 102 includes one or more electronic devices (e.g., 300) networked together (e.g., via a local area network). In some examples, client system 102 includes a computer program or application (comprising instructions executable by one or more processors) for receiving patient data and/or communicating with one or more remote systems (e.g., 112, 126) for the processing of such patient data.

Client system 102 is connected to a network 106 via connection 104. Connection 104 can be used to transmit and/or receive data from one or more other electronic devices or systems (e.g., 112, 126). The network 106 may include any type of network that allows sending and receiving communication signals, such as a wireless telecommunication network, a cellular telephone network, a time division multiple access (TDMA) network, a code division multiple access (CDMA) network, Global System for Mobile communications (GSM), a third-generation (3G) network, fourth-generation (4G) network, a satellite communications network, and other communication networks. The network 106 may include one or more of a Wide Area Network (WAN) (e.g., the Internet), a Local Area Network (LAN), and a Personal Area Network (PAN). In some examples, the network 106 includes a combination of data networks, telecommunication networks, and a combination of data and telecommunication networks. The systems and resources 102, 112 and/or 126 communicate with each other by sending and receiving signals (wired or wireless) via the network 106. In some examples, the network 106 provides access to cloud computing resources (e.g., system 112), which may be elastic/on-demand computing and/or storage resources available over the network 106. The term ‘cloud’ services generally refers to a service performed not locally on a user's device, but rather delivered from one or more remote devices accessible via one or more networks.

Cloud computing system 112 is connected to network 106 via connection 108. Connection 108 can be used to transmit and/or receive data from one or more other electronic devices or systems and can be any suitable type of data connection (e.g., wired, wireless, or any combination of wired and wireless). In some examples, cloud computing system 112 is a distributed system (e.g., remote environment) having scalable/elastic computing resources. In some examples, computing resources include one or more computing resources 114 (e.g., data processing hardware). In some examples, such resources include one or more storage resources 116 (e.g., memory hardware). The cloud computing system 112 can perform processing (e.g., applying one or more machine learning models, applying one or more algorithms) of patient data (e.g., received from client system 102). In some examples, cloud computing system 112 hosts a service (e.g., computer program or application comprising instructions executable by one or more processors) for receiving and processing patient data (e.g., from one or more remote client systems, such as 102). In this way, cloud computing system 112 can provide patient data analysis services to a plurality of health care providers (e.g., via network 106). The service can provide a client system 102 with, or otherwise make available, a client application (e.g., a mobile application, a web-site application, or a downloadable program that includes a set of instructions) executable on client system 102. In some examples, a client system (e.g., 102) communicates with a server-side application (e.g., the service) on a cloud computing system (e.g., 112) using an application programming interface.

In some examples, cloud computing system 112 includes a database 120. In some examples, database 120 is external to (e.g., remote from) cloud computing system 112. In some examples, database 120 is used for storing one or more of patient data, algorithms, machine learning models, or any other information used by cloud computing system 112.

In some examples, system 100 includes cloud computing resource 126. In some examples, cloud computing resource 126 provides external data processing and/or data storage service to cloud computing system 112. For example, cloud computing resource 126 can perform resource-intensive processing tasks, such as machine learning model training, as directed by the cloud computing system 112. In some examples, cloud computing resource 126 is connected to network 106 via connection 124. Connection 124 can be used to transmit and/or receive data from one or more other electronic devices or systems and can be any suitable type of data connection (e.g., wired, wireless, or any combination of wired and wireless). For example, cloud computing system 112 and cloud computing resource 126 can communicate via network 106, and connections 108 and 124. In some examples, cloud computing resource 126 is connected to cloud computing system 112 via connection 122. Connection 122 can be used to transmit and/or receive data from one or more other electronic devices or systems and can be any suitable type of data connection (e.g., wired, wireless, or any combination of wired and wireless). For example, cloud computing system 112 and cloud computing resource 126 can communicate via connection 122, which is a private connection.

In some examples, cloud computing resource 126 is a distributed system (e.g., remote environment) having scalable/elastic computing resources. In some examples, computing resources include one or more computing resources 128 (e.g., data processing hardware). In some examples, such resources include one or more storage resources 130 (e.g., memory hardware). The cloud computing resource 126 can perform processing (e.g., applying one or more machine learning models, applying one or more algorithms) of patient data (e.g., received from client system 102 or cloud computing system 112). In some examples, cloud computing system (e.g., 112) communicates with a cloud computing resource (e.g., 126) using an application programming interface.

In some examples, cloud computing resource 126 includes a database 134. In some examples, database 134 is external to (e.g., remote from) cloud computing resource 126. In some examples, database 134 is used for storing one or more of patient data, algorithms, machine learning models, or any other information used by cloud computing resource 126.

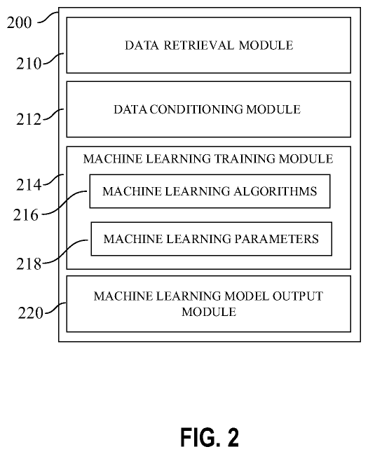

FIG. 2 illustrates an exemplary machine learning system 200 in accordance with some embodiments. In some embodiments, a machine learning system (e.g., 200) is comprised of one or more electronic devices (e.g., 300). In some embodiments, a machine learning system includes one or more modules for performing tasks related to one or more of training one or more machine learning algorithms, applying one or more machine learning models, and outputting and/or manipulating results of machine learning model output. Machine learning system 200 includes several exemplary modules. In some embodiments, a module is implemented in hardware (e.g., a dedicated circuit), in software (e.g., a computer program comprising instructions executed by one or more processors), or some combination of both hardware and software. In some embodiments, the functions described below with respect to the modules of machine learning system 200 are performed by two or more electronic devices that are connected locally, remotely, or some combination of both. For example, the functions described below with respect to the modules of machine learning system 200 can be performed by electronic devices located remotely from each other (e.g., a device within system 112 performs data conditioning, and a device within system 126 performs machine learning training).

In some embodiments, machine learning system 200 includes a data retrieval module 210. Data retrieval module 210 can provide functionality related to acquiring and/or receiving input data for processing using machine learning algorithms and/or machine learning models. For example, data retrieval module 210 can interface with a client system (e.g., 102) or server system (e.g., 112) to receive data that will be processed, including establishing communication and managing transfer of data via one or more communication protocols.

In some embodiments, machine learning system 200 includes a data conditioning module 212. Data conditioning module 212 can provide functionality related to preparing input data for processing. For example, data conditioning can include making a plurality of images uniform in size (e.g., cropping, resizing), augmenting data (e.g., taking a single image and creating slightly different variations (e.g., by pixel rescaling, shear, zoom, rotating/flipping), extrapolating, feature engineering), adjusting image properties (e.g., contrast, sharpness), filtering data, or the like.

In some embodiments, machine learning system 200 includes a machine learning training module 214. Machine learning training module 214 can provide functionality related to training one or more machine learning algorithms, in order to create one or more trained machine learning models.

The concept of “machine learning” generally refers to the use of one or more electronic devices to perform one or more tasks without being explicitly programmed to perform such tasks. A machine learning algorithm can be “trained” to perform the one or more tasks (e.g., classify an input image into one or more classes, identify and classify features within an input image, predict a value based on input data) by applying the algorithm to a set of training data, in order to create a “machine learning model” (e.g., which can be applied to non-training data to perform the tasks). A “machine learning model” (also referred to herein as a “machine learning model artifact” or “machine learning artifact”) refers to an artifact that is created by the process of training a machine learning algorithm. The machine learning model can be a mathematical representation (e.g., a mathematical expression) to which an input can be applied to get an output. As referred to herein, “applying” a machine learning model can refer to using the machine learning model to process input data (e.g., performing mathematical computations using the input data) to obtain some output.

Training of a machine learning algorithm can be either “supervised” or “unsupervised”. Generally speaking, a supervised machine learning algorithm builds a machine learning model by processing training data that includes both input data and desired outputs (e.g., for each input data, the correct answer (also referred to as the “target” or “target attribute”) to the processing task that the machine learning model is to perform). Supervised training is useful for developing a model that will be used to make predictions based on input data. An unsupervised machine learning algorithm builds a machine learning model by processing training data that only includes input data (no outputs). Unsupervised training is useful for determining structure within input data.

A machine learning algorithm can be implemented using a variety of techniques, including the use of one or more of an artificial neural network, a deep neural network, a convolutional neural network, a multilayer perceptron, and the like.

Referring again to FIG. 2, in some examples, machine learning training module 214 includes one or more machine learning algorithms 216 that will be trained. In some examples, machine learning training module 214 includes one or more machine learning parameters 218. For example, training a machine learning algorithm can involve using one or more parameters 218 that can be defined (e.g., by a user) that affect the performance of the resulting machine learning model. Machine learning system 200 can receive (e.g., via user input at an electronic device) and store such parameters for use during training. Exemplary parameters include stride, pooling layer settings, kernel size, number of filters, and the like, however this list is not intended to be exhaustive.

In some examples, machine learning system 200 includes machine learning model output module 220. Machine learning model output module 220 can provide functionality related to outputting a machine learning model, for example, based on the processing of training data. Outputting a machine learning model can include transmitting a machine learning model to one or more remote devices. For example, a machine learning system 200 implemented on electronic devices of cloud computing resource 126 can transmit a machine learning model to cloud computing system 112, for use in processing patient data sent between client system 102 and system 112.

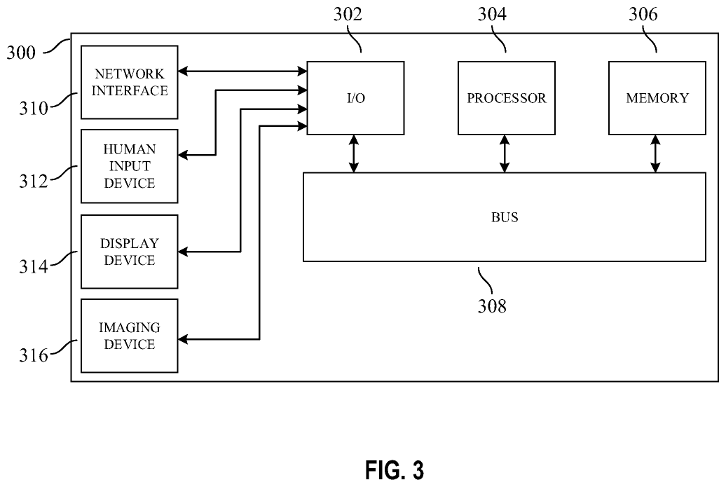

FIG. 3 illustrates exemplary electronic device 300 which can be used in accordance with some examples. Electronic device 300 can represent, for example, a PC, a smartphone, a server, a workstation computer, a medical device, or the like. In some examples, electronic device 300 comprises a bus 308 that connects input/output (I/O) section 302, one or more processors 304, and memory 306. In some examples, electronic device 300 includes one or more network interface devices 310 (e.g., a network interface card, an antenna). In some examples, I/O section 302 is connected to the one or more network interface devices 310. In some examples, electronic device 300 includes one or more human input devices 312 (e.g., keyboard, mouse, touch-sensitive surface). In some examples, I/O section 302 is connected to the one or more human input devices 312. In some examples, electronic device 300 includes one or more display devices 314 (e.g., a computer monitor, a liquid crystal display (LCD), light-emitting diode (LED) display). In some examples, I/O section 302 is connected to the one or more display devices 314. In some examples, I/O section 302 is connected to one or more external display devices. In some examples, electronic device 300 includes one or more imaging device 316 (e.g., a camera, a device for capturing medical images). In some examples, I/O section 302 is connected to the imaging device 316 (e.g., a device that includes a computer-readable medium, a device that interfaces with a computer readable medium).

In some examples, memory 306 includes one or more computer-readable mediums that store (e.g., tangibly embodies) one or more computer programs (e.g., including computer executable instructions) and/or data for performing techniques described herein in accordance with some examples. In some examples, the computer-readable medium of memory 306 is a non-transitory computer-readable medium. At least some values based on the results of the techniques described herein can be saved into memory, such as memory 306, for subsequent use. In some examples, a computer program is downloaded into memory 306 as a software application. In some examples, one or more processors 304 include one or more application-specific chipsets for carrying out the above-described techniques.

### 2. Processes for Differentially Diagnosing Asthma and COPD

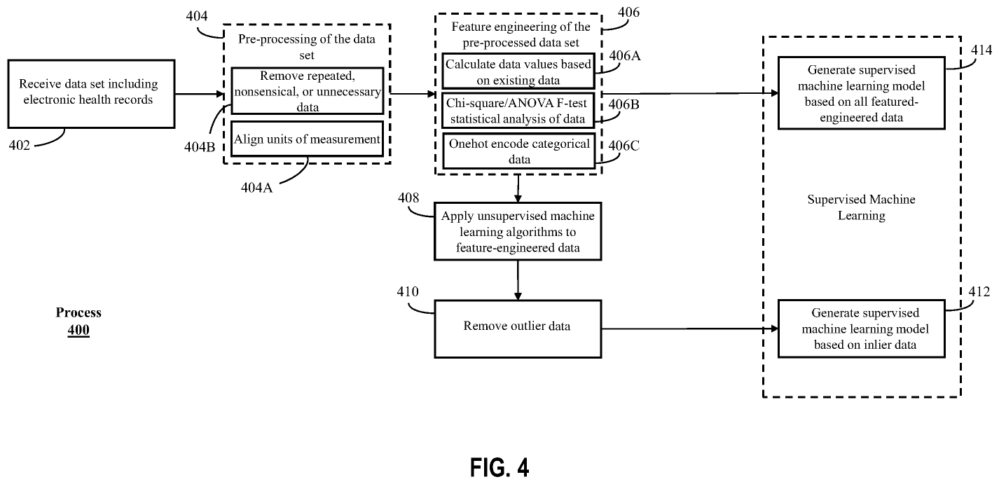

FIG. 4 illustrates an exemplary, computerized process for generating two supervised machine learning models for differentially diagnosing asthma and COPD in a patient. In some examples, process 400 is performed by a system having one or more features of system 100, shown in FIG. 1. For example, one or more blocks of process 400 can be performed by client system 102, cloud computing system 112, and/or cloud computing resource 126.

At block 402, a computing system (e.g., client system 102, cloud computing system 112, and/or cloud computing resource 126) receives a data set (e.g., via data retrieval module 210) including anonymized electronic health records related to asthma and/or COPD from an external source (e.g., database 120 or database 134). In some examples, the external source is a commercially available database. In other examples, the external source is a private Key Opinion Leader (“KOL”) database. The data set includes anonymized electronic health records for a plurality of patients diagnosed with asthma and/or COPD. In some examples, the data set includes anonymized electronic health records for millions of patients diagnosed with asthma and/or COPD. The electronic health records include a plurality of data inputs for each of the plurality of patients. The plurality of data inputs represent patient features, physiological measurements, and other information relevant to diagnosing asthma and/or COPD. The electronic health records further include a diagnosis of asthma and/or COPD for each of the plurality of patients. In some examples, the computing system receives more than one data set including anonymized electronic health records related to asthma and/or COPD from various sources (e.g., receiving a data set from a commercially available database and another data set from a KOL database). In these examples, block 402 further includes the computing system combining the received data sets into a single combined data set.

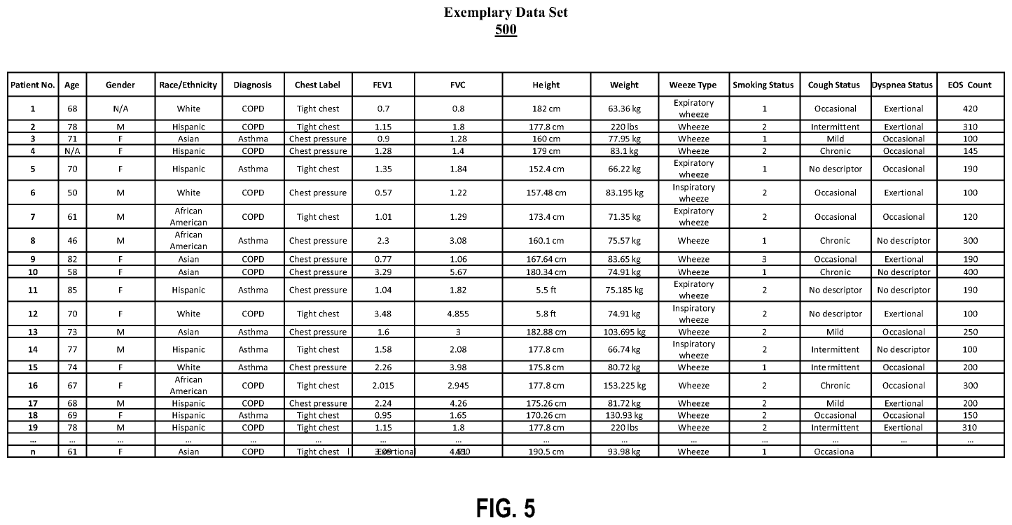

FIG. 5 illustrates a portion of an exemplary data set including anonymized electronic health records for a plurality of patients diagnosed with asthma and/or COPD. Specifically, FIG. 5 illustrates a portion of exemplary data set 500. As shown, exemplary data set 500 includes a plurality of data inputs, as well as an asthma or COPD diagnosis, for Patient 1 through Patient n. Specifically, the plurality of data inputs include patient age, gender (e.g., male or female), race/ethnicity (e.g., White, Hispanic, Asian, African American, etc.), chest label (e.g., tight chest, chest pressure, etc.), forced expiratory volume in one second (FEV1) measurement, forced vital capacity (FVC) measurement, height, weight, smoking status (e.g., number of cigarette packs per year), cough status (e.g., occasional, intermittent, mild, chronic, etc.), dyspnea status (e.g., exertional, occasional, etc.), and Eosinophil (EOS) count. Some data inputs (e.g., cough status, dyspnea status, etc.) have a “No descriptor” value, which represents that a patient has not provided a value for that data input (e.g., if the data input does not apply to the patient).

In some examples, the data set received at block 402 includes more data inputs than those included in exemplary data set 500 for one or more patients of the plurality of patients. Some examples of additional data inputs include (but are not limited to) a patient body mass index (BMI), FEV1/FVC ratio, median FEV1/FVC ratio (e.g., if a patient's FEV1 and FVC has been measured more than once), wheeze status (e.g., coarse, bilateral, slight, prolonged, etc.), wheeze status change (e.g., increased, decreased, etc.), cough type (e.g., regular cough, productive cough, etc.), dyspnea type (e.g., paroxysmal nocturnal dyspnea, trepopnea, platypnea, etc.), dyspnea status change (e.g., improved, worsened, etc.), chronic rhinitis count (e.g., number of positive diagnoses), allergic rhinitis count (e.g., number of positive diagnoses), gastroesophageal reflux disease count (e.g., number of positive diagnoses), location data (e.g., barometric pressure and average allergen count of patient residence), and sleep data (e.g., average hours of sleep per night). Additionally, in some examples, the data set includes image data for one or more patients of the plurality of patients included in the data set (e.g., chest radiographs/x-ray images). In some examples, the data set received at block 402 includes less data inputs than those included in exemplary data set 500 for one or more patients of the plurality of patients.

Returning to FIG. 4, at block 404, the computing system pre-processes the data set received at block 402 (e.g., via data conditioning model 212). In the examples mentioned above where the computing system receives more than one data set at block 402, the computing system pre-process the single combined data set. As shown in FIG. 4, pre-processing the data set at block 404 includes removing repeated, nonsensical, or unnecessary data from the data set at block 404A and aligning units of measurement for data input values included in the data set at block 404B. In some examples, removing repeated, nonsensical, or unnecessary data at block 404A includes removing repeated, nonsensical, and/or unnecessary data inputs for one or more patients of the plurality of patients included in the data set. For example, a data input is unnecessary if the data input has not been identified (e.g., by physicians and research scientists) as being important to the diagnosis of asthma and/or COPD. In some examples, removing repeated, nonsensical, or unnecessary data at block 404A includes entirely removing one or more patients (and all of their corresponding data inputs) from the data set if the data inputs for the one or more patients do not include one or more core data inputs. Some examples of core data inputs include (but are not limited to) patient age, gender, height, and/or weight.

In some examples, aligning units of measurement for data input values included in the data set at block 404B includes converting all data input values to corresponding metric values (where applicable). For example, converting data input values to corresponding metric values includes converting all data input values for patient height in the data set to centimeters (cm) and/or converting all data input values for patient weight in the data set to kilograms (kg).

In some examples, block 404 does not include one of block 404A and block 404B. For example, block 404 does not include block 404A if there is no repeated, nonsensical, or unnecessary data in the data set received at block 402. In some examples, block 404 does not include block 404B if all of the units of measurement for data input values included in the data set received at block 402 are already aligned (e.g., already in metric units).

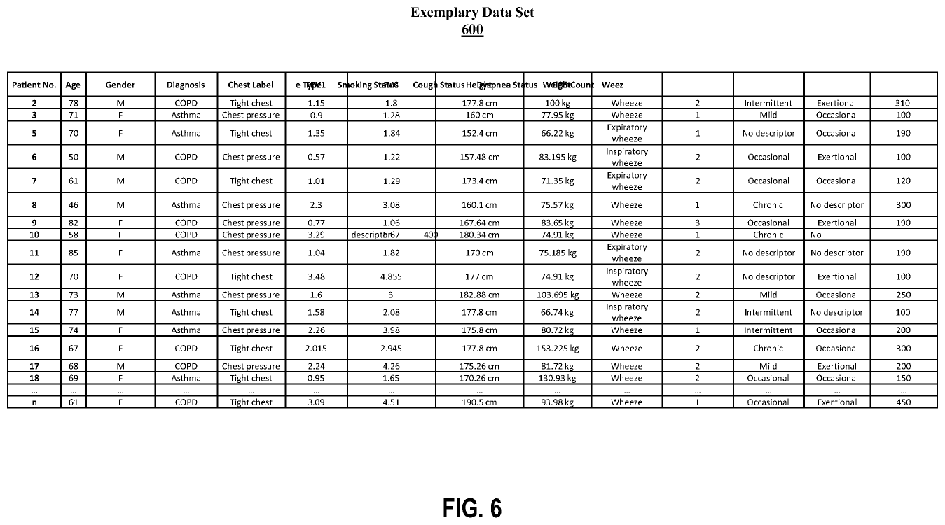

FIG. 6 illustrates a portion of an exemplary data set after pre-processing. Specifically, FIG. 6 illustrates a portion of exemplary data set 600, which is generated by the computing system based on the pre-processing of exemplary data set 500. As shown, the computing system removed all patient race/ethnicity data inputs from exemplary data set 500. In this example, the computing system removed all patient race/ethnicity data inputs from exemplary data set 500 because the computing system determined that patient race/ethnicity is an unnecessary data input. Specifically, the computing system determined that patient race/ethnicity is an unnecessary data input because, in this example, patient race/ethnicity had not been identified (e.g., by physicians and research scientists) as being important to the diagnosis of asthma and/or COPD. Further, the computing system entirely removed Patient 1 and Patient 4 (and all of their corresponding data inputs) from exemplary data set 500. In this example, the computing system removed Patient 1 and Patient 4 from exemplary data set 500 because their data inputs did not include a core data input. Specifically, both patient gender and patient age were core data inputs, but the data inputs for Patient 1 did not include a patient gender data input (e.g., male (M) or female (F)) and the data inputs for Patient 4 did not include a patient age data input.

The computing system also entirely removed Patient 19 (and all of Patient 19's corresponding data inputs) from exemplary data set 500. In this example, the computing system entirely removed Patient 19 from exemplary data set 500 because the computing system determined that Patient 19 was a duplicate of Patient 2 (e.g., all of the data inputs for Patient 19 and Patient 2 were identical and thus Patient 19 was a repeat of Patient 2). Lastly, the computing system aligned the units for the patient weight data input of Patient 2 as well as the patient height data inputs of Patient 11 and Patient 12. Specifically, the computing system converted the values/units for the patient weight data input of Patient 2 from 220 pounds (lb) to 100 kilograms (kg) and the values/units for the patient height data inputs of Patient 11 and Patient 12 from 5.5 feet (ft) and 5.8 ft to 170 centimeters (cm) and 177 cm, respectively.

Returning to FIG. 4, at block 406, the computing system feature-engineers the pre-processed data set generated at block 404 (e.g., via data conditioning model 212). As shown, feature-engineering the pre-processed data set at block 406 includes calculating (e.g., extrapolating) values for one or more new data inputs for one or more patients of the plurality of patients included in the data set based on the values of one or more data inputs of the plurality of data inputs for the one or more patients at block 406A. Some examples of values for the one or more new data inputs that the computing system calculates include (but are not limited to) patient BMI, FEV1/FVC ratio, predicted FEV1, predicted FVC, and/or predicted FEV1/FVC ratio (e.g., a ratio of predicted FEV1 over predicted FVC). In some examples, calculating the values for the one or more new data inputs based on the values of the one or more data inputs of the plurality of data inputs includes calculating the values for the one or more new data inputs based on existing models available within relevant research and/or academic literature (e.g., calculating a value for a predicted patient FEV1 data input based on patient gender and race data input values). In some examples, calculating the values for the one or more new data inputs based on the values of the one or more data inputs of the plurality of data inputs includes calculating the values for the one or more new data inputs based on patient age, gender, and/or race/ethnicity matched averages (e.g., averages provided by physicians and/or research scientists, averages within relevant research and/or academic literature, etc.). In some examples, block 406A further includes the computing system adding the one or more new data inputs for the one or more patients to the data set after calculating the values for the one or more new data inputs.

Feature-engineering the pre-processed data set at block 406 further includes the computing system calculating, at block 406B, chi-square statistics corresponding to one or more categorical data inputs for each of the plurality of patients included in the data set and Analysis of Variance (ANOVA) F-test statistics corresponding to one or more non-categorical data inputs for each of the plurality of patients included in the data set. Categorical data inputs include data inputs having non-numerical data input values. Some examples of non-numerical data input values include (but are not limited to) “tight chest” or “chest pressure” for a patient chest label data input and “intermittent,” “mild,” “occasional,” or “no descriptor” for a patient cough status data input. Non-categorical data inputs include data inputs having numerical data input values.

The computing system utilizes chi-square and ANOVA F-test statistics to measure variance between the values of one or more data inputs included in the data set in relation to asthma or COPD diagnoses included in the data set (e.g., the “target attribute” of the data set). Accordingly, the computing system determines, based on the calculated chi-square and ANOVA F-test statistics, one or more data inputs that are most likely to be independent of class and therefore unhelpful and/or irrelevant for training machine learning algorithms using the data set to predict asthma and/or COPD diagnoses. In other words, the computing system determines one or more data inputs (of the data inputs included in the data set) that have high variance in relation to the asthma or COPD diagnoses included in the data set when compared with other data inputs included in the data set. In some examples, determining the one or more data inputs that are most likely to be independent of class further includes the computing system performing recursive feature elimination with cross-validation (RFECV) based on the data set (e.g., after calculating the chi-square and ANOVA F-test statistics). In some examples, block 406B further includes the computing system removing the one or more data inputs that the computing system determines are most likely to be independent of class for one or more patients of the plurality of patients included in the data set.

Feature-engineering the pre-processed data set at block 406 further includes the computing system one-hot encoding categorical data inputs for each of the plurality of patients included in the data set at block 406C. As described above, categorical data inputs include data inputs having non-numerical data input values. With respect to block 406C, categorical data inputs further include diagnoses of asthma or COPD included in the data set (as a diagnosis of asthma or COPD is a non-numerical value). One-hot encoding is a process by which categorical data input values are converted into a form that can be used to train machine learning algorithms and in some cases improve the predictive ability of a trained machine learning algorithm. Accordingly, one-hot encoding categorical data input values for each of the plurality of patients included in the data set includes converting each of the plurality of patients' non-numerical data input values and diagnosis of asthma or COPD into numerical values and/or binary values representing the non-numerical data input values and asthma or COPD diagnosis. For example, the non-numerical data input values “tight chest” and “chest pressure” for the patient chest label data input are converted to binary values 0 and 1, respectively. Similarly, an asthma diagnosis and a COPD diagnosis are converted to binary values 0 and 1, respectively.

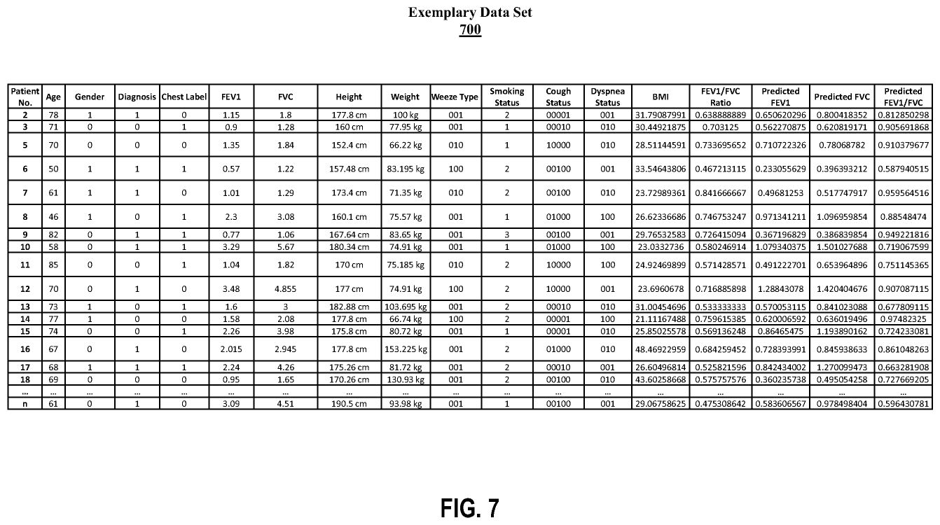

FIG. 7 illustrates a portion of an exemplary data set after feature engineering. Specifically, FIG. 7 illustrates a portion of exemplary data set 700, which is generated by the computing system based on the feature engineering of exemplary data set 600. As shown, the computing system calculated values for five new data inputs for each of the plurality of patients included in exemplary data set 600 (e.g., Patient 2, Patient 3, and Patient 5 through Patient n) and added the new data inputs to exemplary data set 600. Specifically, the computing system calculated values, and added new data inputs for, patient BMI, FEV1/FVC ratio, predicted FEV1, predicted FVC, and predicted FEV1/FVC ratio for each of the plurality of patients include in exemplary data set 600. As explained above, the computing system could have calculated the values for the new data inputs based on (1) the values of one or more data inputs of the plurality of data inputs for each of the plurality of patients, (2) existing models available within relevant research and/or academic literature, and/or (3) patient age and/or gender matched averages (but not race/ethnicity matched averages, as the race/ethnicity data inputs were removed during the pre-processing of exemplary data set 500). For example, the computing system could have determined the values for the patient BMI data input based on the values of the height and weight data inputs for each of the plurality of patients included in exemplary data set 600 and existing models for calculating BMI (e.g., BMI=weight in kg/(height in cm/100)²).

As shown in FIG. 7, the computing system also removed the EOS count data input for each of the plurality of patients included in exemplary data set 600. Specifically, in this example, the computing system calculated chi-square statistics corresponding to the categorical data inputs for each of the plurality of patients included in exemplary data set 600 and ANOVA F-test statistics corresponding to the non-categorical data inputs for each of the plurality of patients included in exemplary data set 600. Then, the computing system determined, based on the calculated ANOVA F-test statistics, that the patient EOS count data input is likely to be independent of class (e.g., relative to the other data inputs) and therefore unhelpful and/or irrelevant for training machine learning algorithms using exemplary data set 600. Note that the computing system made this determination regarding the EOS count data input based on the ANOVA F-test statistics because EOS count is a non-categorical data input. After determining that the patient EOS count data input is likely to be independent of class, the computing system removed the EOS count data input for each of the plurality of patients include in exemplary data set 600.

Lastly, as shown in FIG. 7, the computing system also one-hot encoded categorical data input values for each of the plurality of patients included in exemplary data set 600. Specifically, the computing system converted the non-numerical values for the patient gender, chest label, wheeze type, cough status, and dyspnea status data inputs for each of the plurality of patients included in exemplary data set 600 into binary values representing the non-numerical values. For example, with respect to the patient chest label data input, the computing device converted all “tight chest” values to a binary value of “0” and all “chest pressure” values to a binary value of “1.” As another example, with respect to the wheeze type data input, the computing device converted all “Wheeze” values to a binary value of “001,” all “Expiratory wheeze” values to a binary value of “010,” and all “Inspiratory wheeze” values to a binary value of “100.” Moreover, the computing system one-hot encoded the diagnosis of asthma or COPD for each of the plurality of patients included in exemplary data set 400 by converting all “asthma” values to a binary value of “0” and all “COPD” values to a binary value of “1.”

Returning to FIG. 4, at block 408, the computing system applies two unsupervised machine learning algorithms (e.g., included in machine learning algorithms 216) to the feature-engineered data set generated at block 406 (e.g., via machine learning training module 214). The first unsupervised machine learning algorithm that the computing system applies to the data set is a Uniform Manifold Approximation and Projection (UMAP) algorithm. The reduced-dimension representations of the data set include a reduced-dimension representation of the data input values for each of the plurality of patients included in the data set in the form of one or more coordinates. In some examples, applying a UMAP algorithm to the data set generates a two-dimensional representation of the data input values for each of the plurality of patients included in the data set in the form of two-dimensional coordinates (e.g., x and y coordinates). In other examples, applying a UMAP algorithm to the data set generates a reduced-dimension representation of the data input values for each of the plurality of patients included in the data set that has more than two dimensions (e.g., a three-dimensional representation). In some examples, the computing system applies one or more other algorithms and/or techniques to non-linearly reduce the data set's number of dimensions and generate reduced-dimension representations of the data set instead of applying the UMAP algorithm discussed above. Some examples of such algorithms and/or techniques include (but are not limited to) Isomap (or other non-linear dimensionality reduction methods), robust feature scaling followed by Principal Component Analysis (PCA) or Linear Discriminant Analysis (LDA), and normal feature scaling followed by PCA or LDA.

In some examples, after generating a reduced-dimension representation of the data input values for each of the plurality of patients included in the data set (e.g., in the form of one or more coordinates), the computing system adds the reduced-dimension representation of the data input values to the data set as one or more new data inputs for each of the patients. For example, in the example above wherein the computing system generates a two-dimensional representation of the data input values for each patient included in the data set in the form of two-dimensional coordinates, the computing system subsequently adds a new data input for each coordinate of the two dimensional coordinates for each patient of the plurality of patients.

Further, after applying the UMAP algorithm to the data set, the computing system generates a UMAP model (e.g., a machine learning model artifact) representing the non-linear reduction of the feature-engineered data set's number of dimensions (e.g., via machine learning model output module 220). Then, as will be described in greater detail below, if the computing system applies the generated UMAP model to, for example, a set of patient data including a plurality of data inputs corresponding to a patient not included in the feature-engineered data set, the computing system determines (based on the application of the UMAP model) a reduced-dimension representation of the data input values for the patient not included in the data set. Specifically, the computing system determines the reduced-dimension representation of the data input values for the patient not included in the feature-engineered data set by non-linearly reducing the set of patient data in the same manner that the computing system reduced the feature-engineered data set's number of dimensions.

After generating a reduced-dimension representation of the data input values for each of the plurality of patients included in the feature-engineered data set (e.g., in the form of one or more coordinates), the computing system applies a Hierarchical Density-Based Spatial Clustering of Applications with Noise (HDB SCAN) unsupervised machine learning algorithm to the reduced-dimension representations of the data input values. Applying an HDBSCAN algorithm to the reduced-dimension representation of the data set clusters one or more patients of the plurality of patients included in the data set into one or more clusters (such as groups) of patients based on the reduced-dimension representation of the one or more patients' data input values and one or more threshold similarity/correlation requirements (discussed in greater detail below). Each generated cluster of patients of the one or more generated clusters of patients includes two or more patients having similar/correlated reduced-dimension representations of their data input values (e.g., similar/correlated coordinates). The one or more patients that are clustered into one cluster of patients are referred to as “inliers” and/or “phenotypic hits.” In some examples, the computing system applies one or more other algorithms to the data set to cluster one or more patients of the plurality of patients included in the data set into one or more clusters of patients instead of applying the HDB SCAN algorithm mentioned above. Some examples of such algorithms include (but are not limited to) a K-Means clustering algorithm, a Mean-Shift clustering algorithm, and a Density-Based Spatial Clustering of Applications with Noise (DBSCAN) algorithm.

Note, in some examples, one or more patients of the plurality of patients included in the data set will not be clustered into a cluster of patients. The one or more patients that are not clustered into a cluster of patients are referred to as “outliers” and/or “phenotypic misses.” For example, the computing system will not cluster a patient into a cluster of patients if the computing system determines (based on the application of the HDBSCAN algorithm to the reduced-dimension representation of the data set) that reduced-dimension representation of the patient's data input values do not meet one or more threshold similarity/correlation requirements.

In some examples, the one or more threshold similarity/correlation requirements include a requirement that each coordinate of a reduced-dimension representation of a patient's data input values (e.g., x, y, and z coordinates for a three-dimensional representation) be within a certain numerical range in order to be clustered into a cluster of patients. In some examples, the one or more threshold similarity/correlation requirements include a requirement that at least one coordinate of a reduced-dimension representation of a patient's data input values be within a certain proximity to a corresponding coordinate of reduced-dimension representations of one or more other patients' data input values. In some examples, the one or more threshold similarity/correlation requirements include a requirement that all coordinates of a reduced-dimension representation of a patient's data input values be within a certain proximity to corresponding coordinates for reduced-dimension representations of a minimum number of other patients included in the data set. In some examples, the one or more threshold similarity/correlation requirements include a requirement that all coordinates of a reduced-dimension representation of a patient's data input values be within a certain proximity to a cluster centroid (e.g., a center point of a cluster). In these examples, the computing system determines a cluster centroid for each of the one or more clusters that the computing system generates based on the application of the HDBSCAN algorithm to the data set.

In some examples, the one or more threshold similarity/correlation requirements are predetermined. In some examples, the computing system generates the one or more threshold similarity/correlation requirements based on the application of the HDBS CAN algorithm to the reduced-dimension representation of the data set or the data set itself.

After applying the HDBSCAN algorithm to the reduced-dimension representations of the data input values for each of the plurality of patients included in the data set, the computing system generates (e.g., via machine learning model output module 220) an HDBSCAN model representing a cluster structure of the data set (e.g., a machine learning model artifact representing the one or more generated clusters and relative positions of inliers and outliers included in the data set). Then, as will be described in greater detail below, if the computing system applies the generated HDBSCAN model to, for example, a reduced-dimension representation of data input values included in a set of patient data for a patient not include in the data set, the computing system determines (based on the application of the HDBSCAN model) whether the patient falls within one of the one or more generated clusters corresponding to the plurality of patients included in the data set. In other words, the computing device determines, based on the application of the HDBSCAN model to the reduced-dimension representation of data input values for the patient, whether each of the patients is an inlier/phenotypic hit or outlier/phenotypic miss with respect to the one or more generated clusters corresponding to the plurality of patients included in the data set.

In some examples, at step 408, the computing system applies one or more Gaussian mixture model algorithms to the feature-engineered data set instead of the UMAP and HDBSCAN algorithms. A Gaussian mixture model algorithm, like the UMAP and HDBSCAN algorithms, is an unsupervised machine learning algorithm. Further, similar to applying UMAP and HDBSCAN algorithms to the feature-engineered data set, applying one or more Gaussian mixture model algorithms to the data set allows the computing system to classify patients included in the data set as inliers or outliers. Specifically, the computing system determines a covering manifold (e.g., a surface manifold) for the data set based on the application of the one or more Gaussian mixture model algorithms to the data set. Then, the computing system determines whether a patient is an inlier or an outlier based on whether the patient falls within the covering manifold (e.g., a patient is an inlier if the patient falls within the covering manifold). However, the Gaussian mixture model algorithms provide an additional benefit in that their rejection probability is tunable, which in turn allows the computing system to adjust the probability that a patient included in the data set will fall within the covering manifold and thus the probability that a patient will be classified as an outlier.

In some examples, at step 408, the computing system stratifies the feature-engineered data set based on a specific data input included in the data set (e.g., gender, smoking status, FEV1, FEV1/FVC ratio, BMI, number of symptoms, or weight) and then applies a separate Gaussian mixture model algorithm to each stratified subset of the data set. For example, if the computing system stratifies the data set based on gender, the computing system will subsequently apply one Gaussian mixture model algorithm only to male patients included in the data set and apply another Gaussian mixture model algorithm only to female patients included in the data set. In addition to classifying patients included in the stratified subsets as inliers or outliers, stratifying the data set as described above allows the computing system to account for data input values that are dependent upon other data input values included in the feature-engineered data set. For example, because FEV1 and FEV1/FVC ratio values are highly dependent upon gender (e.g., a normal FEV1 measurement for women would be abnormal for men), applying separate Gaussian mixture model algorithms to a subset of female patients and a subset of male patients allows the computing system to account for the FEV1 and FEV1/FVC ratio dependencies when classifying patients as inliers or outliers (e.g., when applying the trained Gaussian mixture model to patient data). This in turn improves the computing system's classification of patients as inliers or outliers (e.g., increased classification accuracy and specificity).

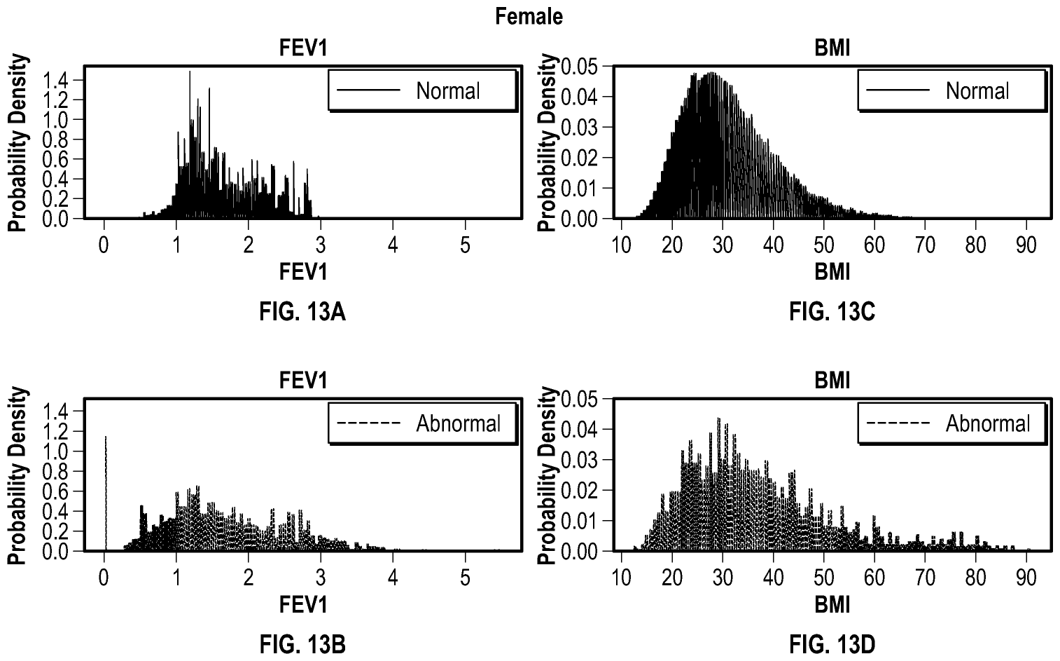

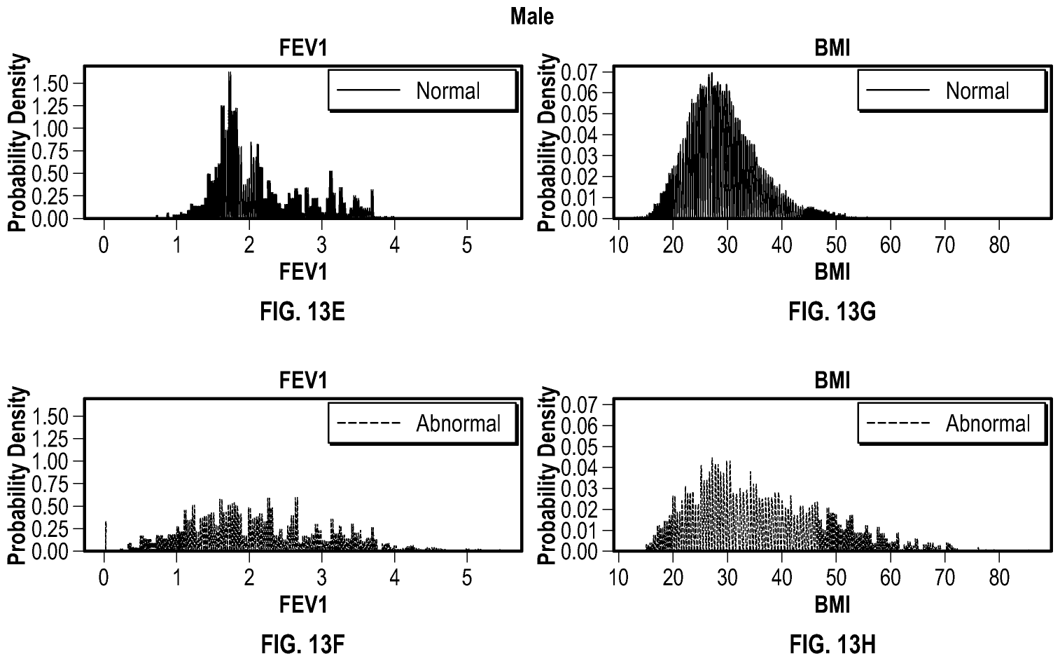

For example, FIGS. 13A-H illustrate bar graphs representing exemplary inlier and outlier classification results based on the application of Gaussian mixture models to subsets of a feature-engineered test set of patient data stratified based on gender. Specifically, FIGS. 13A-D illustrate bar graphs representing inlier (i.e., “Abnormal”) and outlier (i.e., “Normal”) classification results corresponding to the application of a Gaussian mixture model (trained using a training data set of patients that only included data for female patients) to female patients included in the test set of patient data. FIGS. 13E-H illustrate bar graphs representing inlier and outlier classification results (also referred to in the graphs as “Abnormal” and “Normal,” respectively) corresponding to the application of a Gaussian mixture model (trained using a training data set of patients that only included data for male patients) to male patients included in the test set of patient data. Further, the bar graphs illustrated in FIGS. 13A-H correspond to specific data inputs included in the test set of patient data (specifically, FEV1 for FIGS. 13A, 13B, 13E, and 13F; BMI for FIGS. 13C, 13D, 13G, and 13H) such that the graphs illustrate the distribution of values for the specific data input for inlier and outlier patients. As shown, outlier patients (those referred to as “Normal”) are less likely to have irregular/abnormal values for their data input values (in this case FEV1 and BMI), which is why their data input value distributions shown in FIGS. 13A, 13C, 13E, and 13G are more uniform and less scattered than the data input values of the inlier patients (those referred to as “Abnormal”). This is due in part to the computing system's application of Gaussian mixture models that were trained with training data subsets stratified based on gender, which allowed the computing system to account for the differences in data input values that are dependent on gender when classifying patients included in the test set as inliers or outliers.

At block 410, the computing system generates (e.g., via data conditioning module 212) an inlier data set by removing the outliers/phenotypic misses (e.g., the one or more patients of the plurality of patients included in the data set that are not clustered into a cluster of patients) from the data set. Specifically, the computing system entirely removes the outliers/phenotypic misses (and all of their corresponding data inputs) from the data set such that the only patients remaining in the data set are the patients that the computing system clustered into one of the one or more clusters of patients generated at block 408 (e.g., the inliers/phenotypic hits).

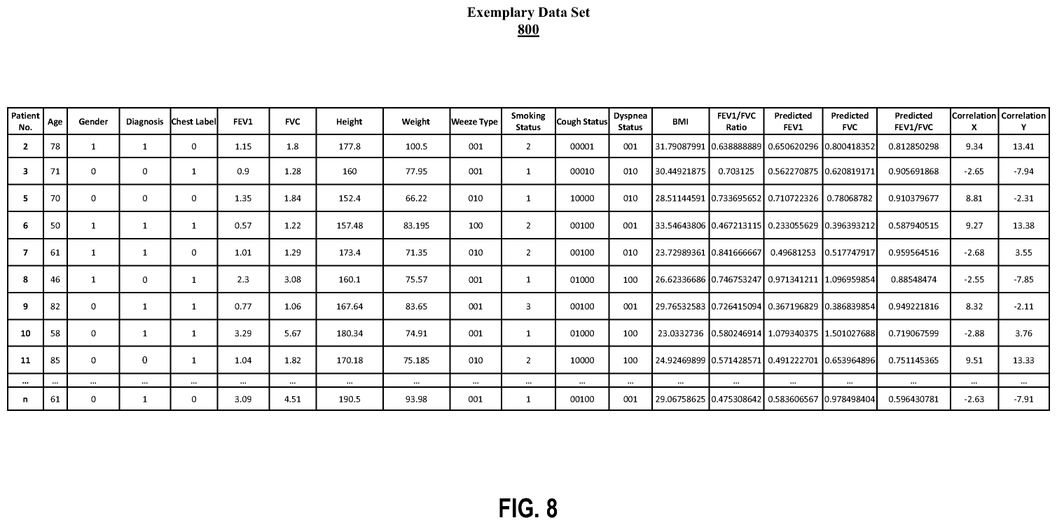

FIG. 8 illustrates a portion of an exemplary data set after the application of two unsupervised machine learning algorithms to the exemplary data set and the removal of all outliers/phenotypic misses from the exemplary data set. Specifically, FIG. 8 illustrates exemplary data set 800, which is generated by the computing system after (1) applying a UMAP algorithm to exemplary data set 700 to generate a two-dimensional representation of the data input values for each patient included in exemplary data set 700 in the form of two-dimensional coordinates, (2) adding the two-dimensional representation of the data input values for each patient to exemplary data set 700 as two new data inputs for each of the patients (e.g., Correlation X and Correlation Y), (3) applying an HDBSCAN algorithm to the two-dimensional representations of the patients' data input values to cluster a plurality of patients included in exemplary data set 700 into a plurality of clusters of patients, and (4) removing a plurality of outliers/phenotypic misses. In this example, of the patients illustrated in the portion of exemplary data set 700 in FIG. 7, the computing system removed Patient 12 through Patient 18 of exemplary data set 700 based on a determination that that the two-dimensional coordinates for each of those patients did not satisfy one or more threshold similarity/correlation requirements. In other words, the computing system removed Patient 12 through Patient 18 because they were not clustered into a cluster of patients and thus were outliers/phenotypic misses. Further, the computing system did not remove Patient 2, Patient 3, Patients 5-11, and Patient n from exemplary data set 700 based on a determination that the two-dimensional coordinates for each of those patients did satisfy the one or more threshold similarity/correlation requirements In other words, the computing system did not remove Patient 2, Patient 3, Patients 5-11, and Patient n because they were each clustered into a cluster of patients and thus were inliers/phenotypic hits.

For example, as shown in FIG. 8, the computing system clustered each of Patient 2, Patient 3, Patients 5-11, and Patient n into one of four clusters based on the one or more threshold similarity/correlation requirements. Specifically, the first cluster of patients includes Patient 2 (e.g., 9.34 (X) and 13.41 (Y)), Patient 6 (e.g., 9.27 (X) and 13.38 (Y)), and Patient 11 (e.g., 9.51 (X) and 13.33 (Y)). The second cluster of patients includes Patient 3 (e.g., −2.65 (X) and −7.94 (Y)), Patient 8 (e.g., −2.55 (X) and −7.85 (Y)), and Patient n (e.g., −2.63 (X) and −7.91 (Y)). The third cluster of patients includes Patient 5 (e.g., 8.81 (X) and −2.31 (Y)) and Patient 9 (e.g., 8.32 (X) and −2.11 (Y)). Lastly, the fourth cluster of patients includes Patient 7 (e.g., −2.68 (X) and 3.55 (Y)) and Patient 10 (e.g., −2.88 (X) and 3.76 (Y)).

Returning to FIG. 4, at block 412, the computing system generates a supervised machine learning model (e.g., via machine learning model output module 220) by applying a supervised machine learning algorithm (e.g., included in machine learning algorithms 216) to the inlier data set generated at block 410 (e.g., via machine learning training module 214). Some examples of the supervised machine learning algorithm applied to the inlier data set include (but are not limited to) a supervised machine learning algorithm generated using XGBoost, PyTorch, scikit-learn, Caffe2, Chainer, Microsoft Cognitive Toolkit, or TensorFlow. Applying the supervised machine learning algorithm to the inlier data set includes the computing system labeling the asthma/COPD diagnosis for each of the patients included in the inlier data set as a target attribute and subsequently training the supervised machine learning algorithm using the inlier data set. As will be discussed below, a target attribute represents the “correct answer” that the supervised machine learning algorithm is trained to predict. Thus, in this case, the supervised machine learning algorithm is trained using the inlier data set (e.g., the data inputs of the inlier data set) so that the supervised machine learning algorithm may learn to predict an asthma and/or COPD diagnosis when provided with data similar to the inlier data set (e.g., patient data including a plurality of data inputs). In some examples, applying the supervised machine learning algorithm to the inlier data set includes the computing system dividing the inlier data set into a first portion (referred to herein as an “inlier training set”) and a second portion (referred to herein as an “inlier validation set”), labeling the asthma/COPD diagnosis for each of the one or more patients included in the inlier training set as a target attribute, and training the supervised machine learning algorithm using the inlier training set. For example, an inlier training set includes one or more patients included in the inlier data set and all of the one or more patients' data inputs and corresponding asthma/COPD diagnoses.

After training the supervised machine learning algorithm, the computing system generates a supervised machine learning model (e.g., a machine learning model artifact). Generating the supervised machine learning model includes the computing system determining, based on the training of the one or more supervised machine learning algorithms, one or more patterns that map the data inputs of the patients included in the inlier data set to the patients' corresponding asthma/COPD diagnoses (e.g., the target attribute). Thereafter, the computing system generates the supervised machine learning model representing the one or more patterns (e.g., a machine learning model artifact representing the one or more patterns). As will be discussed in greater detail below, the computing system uses the generated supervised machine learning model to predict an asthma and/or COPD diagnosis when provided with data similar to the inlier data set (e.g., patient data including a plurality of data inputs).

In the examples where the inlier data set is divided into an inlier training set and an inlier validation set, generating the supervised machine learning model further includes the computing system validating the supervised machine learning model (generated by applying the supervised machine learning algorithm to the inlier training set) using the inlier validation set. Validating a supervised machine learning model assess the supervised machine learning model's ability to accurately predict a target attribute when provided with data similar to the data used to train the supervised machine learning algorithm that generated the supervised machine learning model. In these examples, the computing system validates the supervised machine learning model to assess the supervised machine learning model's ability to accurately predict an asthma and/or COPD diagnosis when applied to patient data that is similar to the inlier data set used during the training process described above (e.g., patient data including a plurality of data inputs).

There are various types of supervised machine learning model validation methods. Some examples of the types of validation include k-fold cross validation, stratified k-fold cross validation, leave-p-out cross validation, or the like. In some examples, the computing system uses one type of validation to validate the supervised machine learning model (generated by applying the supervised machine learning algorithm to the inlier training set). In other examples, the computing system uses more than one type of validation to validate the supervised machine learning model. Further, in some examples, the number of patients in the inlier training set, the number of patients in the inlier validation set, the number of times the supervised machine learning algorithm is trained, and/or the number of times the supervised machine learning model is validated, are based on the type(s) of validation the computing system uses during the validation process.

Validating the supervised machine learning model includes the computing system removing the asthma/COPD diagnosis for each patient included in the inlier validation set, as that is the target attribute that the supervised machine learning model predicts. After removing the asthma/COPD diagnosis for each patient included in the inlier validation set, the computing system applies the supervised machine learning model to the data input values of the patients included in the inlier validation set, such that the supervised machine learning model determines an asthma and/or COPD diagnosis prediction for each of the patients based on each of the patient's data input values. After, the computing system evaluates the supervised machine learning model's ability to predict an asthma and/or COPD diagnosis, which includes the computing system comparing the patients' determined asthma and/or COPD diagnosis predictions to the patients' true asthma/COPD diagnoses (e.g., the diagnoses that were removed from the inlier validation set). In some examples, the computing system's method for evaluating the supervised machine learning model's ability to predict an asthma and/or COPD diagnosis is based on the type(s) of validation used during the validation process.

In some examples, evaluating the supervised machine learning model's ability to predict an asthma and/or COPD diagnosis includes the computing system determining one or more classification performance metrics representing the predictive ability of the supervised machine learning models. Some examples of the one or more classification performance metrics include an F1 score (also known as an F-score or F-measure), a Receiver Operating Characteristic (ROC) curve, an Area Under Curve (AUC) metric (e.g., a metric based on an area under an ROC curve), a log-loss metric, an accuracy metric, a precision metric, a specificity metric, and a recall metric (also known as a sensitivity metric). In some examples, the computing system iteratively performs the above training and validation processes (e.g., using the inlier training set and inlier validation set, or variations thereof) until the one or more determined classification performance metric satisfies one or more corresponding predetermined classification performance metric thresholds. In these examples, the supervised machine learning model generated by the computing system is the supervised machine learning model associated with one or more classification performance metrics that each satisfy the one or more corresponding predetermined classification performance metric thresholds.

In some examples, validating the supervised machine learning model further includes the computing system tuning/optimizing hyperparameters for the supervised machine learning model (e.g., using techniques specific to the specific supervised machine learning algorithm used to generate the supervised machine learning model). Tuning/optimizing a supervised machine learning model's hyperparameters (also referred to as “deep optimization”), as opposed to maintaining a supervised machine learning model's default hyperparameters (also referred to as “basic optimization”), optimizes the supervised machine learning model's performance and thus improves its ability to make accurate predictions (e.g., improves the model's performance metrics, such as the model's accuracy, sensitivity, etc.).

For example, Table (1) below includes asthma and/or COPD prediction results (e.g., percent of true labels/diagnoses correctly predicted) based on the application of the supervised machine learning model to a test set of patient data when the hyperparameters for the supervised machine learning model were not tuned/optimized during the validation of the model (i.e., basic optimization). On the other hand, Table (2) below includes asthma and/or COPD prediction results (e.g., percent of true labels/diagnoses correctly predicted) based on the application of the supervised machine learning model to the same test set of patient data when the hyperparameters for the supervised machine learning model were tuned/optimized during the validation of the model (i.e., deep optimization). As shown, while the basic optimization supervised machine learning model predicted asthma, COPD, and asthma and COPD (“ACO”) with fairly high accuracy and sensitivity, the accuracy and sensitivity of the deep optimization supervised machine learning model was even higher.

TABLE 1
Table (1): Results of applying a supervised machine learning
model (basic optimization) to a test set of patient data
including data input values for 61,735 patients.

| | Number of | Predicted | Predicted | Predicted |
|---|---|---|---|---|
| True | Patients | ACO | Asthma | COPD |
| Label/ | with True | Diagnosis | Diagnosis | Diagnosis |
| Diagnosis | Label/Diagnosis | Percentage | Percentage | Percentage |
| ACO | 4,116 | 53.57% | 4.74% | 41.69% |
| Asthma | 21,562 | 0.24% | 97.27% | 2.49% |
| COPD | 36,057 | 0.63% | 1.55% | 97.83% |

TABLE 2
Table (2): Results of applying a supervised machine learning
model (deep optimization) to a test set of patient data
including data input values for 61,735 patients.

| | Number of | Predicted | Predicted | Predicted |
|---|---|---|---|---|
| True | Patients | ACO | Asthma | COPD |
| Label/ | with True | Diagnosis | Diagnosis | Diagnosis |
| Diagnosis | Label/Diagnosis | Percentage | Percentage | Percentage |
| ACO | 4,116 | 77.55% | 3.89% | 18.56% |
| Asthma | 21,562 | 0.26% | 98.12% | 1.63% |
| COPD | 36,057 | 0.65% | 1.19% | 98.16% |

In some examples, after validating the supervised machine learning model (and, in some examples, after determining one or more performance metrics corresponding to the supervised machine learning model), the computing system performs feature selection based on the data inputs included in the inlier data set to narrow down the most important data inputs with respect to predicting asthma and/or COPD (e.g., the data inputs that have the greatest impact on the supervised machine learning model's diagnosis predictions). Specifically, the computing system determines the importance of the data inputs included in the inlier data set using one or more feature selection techniques such as recursive feature elimination, Pearson correlation filtering, chi-squared filtering, Lasso regression, and/or tree-based selection (e.g., Random Forest). For example, after performing feature selection for the basic optimization and deep optimization supervised machine learning models discussed above with reference to Table (1) and Table (2), the computing system determined that the most important data inputs included in the inlier data set used to train the two supervised machine learning models were FEV1/FVC ratio, FEV1, cigarette packs smoked per year, patient age, dyspnea incidence, whether the patient is a current smoker, patient BMI, whether the patient is diagnosed with allergic rhinitis, wheeze incidence, cough incidence, whether the patient is diagnosed with chronic rhinitis, and if the patient has never smoked before. In some examples, after the computing system determines the most important data inputs via feature selection, the computing system retrains and revalidates the supervised machine learning model using a reduced inlier training data set and a reduced inlier validation set that only includes values for the data inputs that were determined to be most important. In this manner, the computing system generates a supervised machine learning model that can accurately predict asthma and/or COPD diagnoses based on a reduced number of data inputs. This in turn increases the speed at which the supervised machine learning algorithm can make accurate predictions, as there is less data (i.e., less data input values) that the supervised machine learning algorithm needs to process when determining its diagnosis predictions.

Generating an inlier data set (e.g., in accordance with the processes of block 408) and subsequently generating a supervised machine learning model based on the application of a supervised machine learning algorithm to the inlier data set provides several advantages over simply generating a supervised machine learning model by applying a supervised machine learning algorithm to a larger data set that includes inliers/phenotypic hits and outliers/phenotypic misses. For example, because the inlier data set only includes patients having similar/correlated data input values, the computing system is able to generate a supervised machine learning model that predicts an asthma and/or COPD diagnosis with very high accuracy when applied to a patient having similar/correlated data input values to those of the inlier patients.

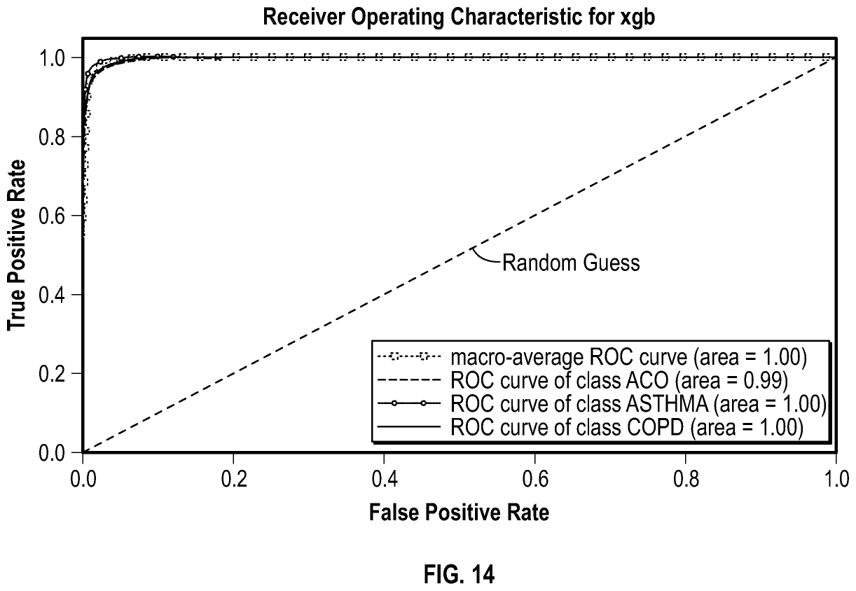

For example, FIG. 14 illustrates a receiver operating characteristic curve representing asthma and/or COPD classification results from the application of the supervised machine learning model (trained using an inlier data set of patients) to a test set of patient data. Further, Table (3) below includes asthma and/or COPD prediction results (e.g., percent of true labels/diagnoses correctly and incorrectly predicted) based on the application of a supervised machine learning model (trained using an inlier data set of patients) to a test set of patient data. In particular, the supervised machine learning model for both FIG. 14 and Table (3) is the same supervised machine learning model, and it was trained using an inlier training data set generated by applying the Gaussian mixture models described above with respect to FIGS. 13A-H to a feature-engineered training data set. As shown in both FIG. 14 and Table (3), the supervised machine learning model was able to classify patients included in the test set of patient data as having asthma, COPD, or asthma and COPD (“ACO”) with very high AUC (area under the ROC curve) metrics and accuracy. As mentioned above, the supervised machine learning model's highly accurate classifications are due, at least in part, to the fact that the supervised machine learning model was trained using an inlier data set instead of, for example, a data set that includes both inlier and outlier patients.

TABLE 3
Table (3): Results of applying a supervised machine learning
model (trained using an inlier data set of patients) to a test set
of patient data including data input values for 11,614 patients.

| | Number of | Predicted | Predicted | Predicted |
|---|---|---|---|---|
| True | Patients | ACO | Asthma | COPD |
| Label/ | with True | Diagnosis | Diagnosis | Diagnosis |
| Diagnosis | Label/Diagnosis | Percentage | Percentage | Percentage |
| ACO | 3,820 | 95.05% | 1.96% | 2.98% |
| Asthma | 3,891 | 1.41% | 97.94% | 0.64% |
| COPD | 3,903 | 3.56% | 0.95% | 95.49% |

At block 414, the computing system generates a supervised machine learning model (e.g., via machine learning model output module 220) by applying a supervised machine learning algorithm (e.g., included in machine learning algorithms 216) to the feature-engineered data set generated at block 406 (e.g., via machine learning training module 214). Block 414 is identical to block 412 except that the computing system applies a supervised machine learning algorithm to a different data set at each block. For example, at block 412, the computing system applies a supervised machine learning algorithm to an inlier data set (generated by the application of one or more unsupervised machine learning algorithms to the feature-engineered data set generated at block 406) whereas at block 414, the computing system applies the same supervised machine learning algorithm directly to a feature-engineered data set after the feature-engineered data set is generated at block 406. In some examples, the computing system uses a different supervised machine learning algorithm at block 412 and block 414. For example, the computing system applies a first supervised machine learning algorithm to the inlier data set at block 412 and a second supervised machine learning algorithm to the feature-engineered data set at block 414.

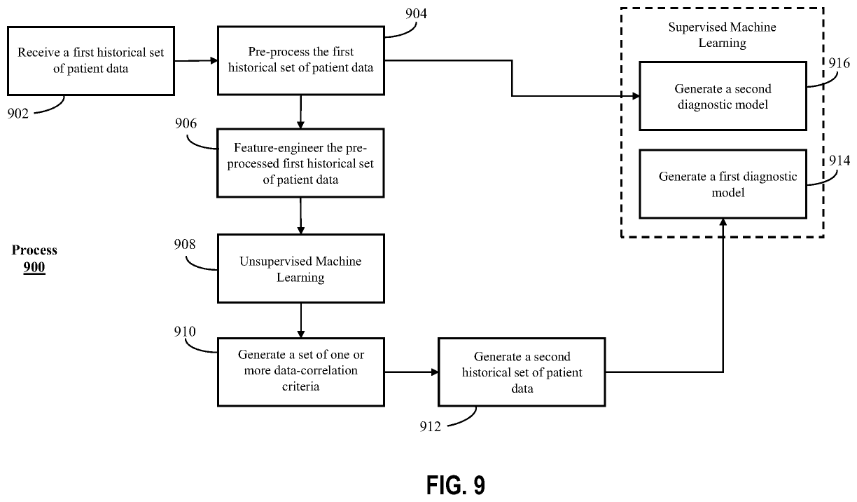

FIG. 9 illustrates an exemplary, computerized process for generating a first diagnostic model and a second diagnostic model for differentially diagnosing asthma and COPD in a patient. In some examples, process 900 is performed by a system having one or more features of system 100, shown in FIG. 1. For example, the blocks of process 900 can be performed by client system 102, cloud computing system 112, and/or cloud computing resource 126.

At block 902, a computing system (e.g., client system 102, cloud computing system 112, and/or cloud computing resource 126) receives a first historical set of patient data (e.g., exemplary data set 500) (e.g., as described above with reference to block 402 of FIG. 4). The first historical set of patient data includes data from a first plurality of patients having one or more phenotypic differences regarding patient features and/or one or more respiratory conditions. In some examples, the phenotypic differences include data regarding one or more respiratory conditions. In some examples, the data regarding one or more respiratory conditions includes a true diagnosis of asthma, COPD, both asthma and COPD, or neither asthma nor COPD. In these examples, a true diagnosis is a diagnosis that has been confirmed by one or more physicians and/or research scientists.

At block 904, the computing system pre-processes the first historical set of patient data received at block 902 (e.g., as described above with reference to block 404 of FIG. 4) and generates a pre-processed first historical set of patient data (e.g., exemplary data set 600). At block 906, the computing system feature-engineers the pre-processed first historical set of patient data (e.g., as described above with reference to block 406 of FIG. 4) and generates a feature-engineered first historical set of patient data (e.g., exemplary data set 700).

At block 908, the computing system applies one or more unsupervised machine learning algorithms to the feature-engineered first historical set of patient data (e.g., as described above with reference to block 408 of FIG. 4). In some examples, the computing system applies one or more unsupervised machine learning algorithms to one or more stratified subsets of the feature-engineered first historical set of patient data (e.g., stratified based on gender, smoking status, FEV1, FEV1/FVC ratio, BMI, number of symptoms, or weight).

At block 910, the computing system generates a set of one or more data-correlation criteria based on the application of the one or more unsupervised machine learning algorithms (e.g., a IJMAP algorithm, HDBSCAN algorithm, and/or Gaussian mixture model algorithm) to the feature-engineered first historical set of patient data. In some examples, at block 910, the computing system generates a set of one or more data-correlation criteria based on the application of the one or more unsupervised machine learning algorithms to one or more stratified subsets of the feature-engineered first historical set of patient data.

In some examples, the set of one or more data-correlation criteria include one or more unsupervised machine learning models (e.g., one or more unsupervised machine learning model artifacts (e.g., e.g., a UMAP model, HDBSCAN model, and/or Gaussian mixture model)) generated by the computing system based on the application of the one or more unsupervised machine learning algorithms to the feature-engineered first historical set of patient data or to one or more stratified subsets of the feature-engineered first historical set of patient data (e.g., as described above with reference to block 408 of FIG. 4). In some examples, the set of one or more data-correlation criteria includes a requirement that a patient fall within in a cluster of one or more clusters of patients generated by applying the one or more unsupervised machine learning algorithms to the feature-engineered first historical set of patient data. In other examples, the set of one or more data-correlation criteria includes a requirement that a patient fall within a covering manifold of patients generated by applying the one or more unsupervised machine learning algorithms to the feature-engineered first historical set of patient data (or to a stratified subset of the feature-engineered first historical set of patient data (e.g., stratified based on gender, smoking status, FEV1, FEV1/FVC ratio, BMI, number of symptoms, or weight)).

At block 912, the computing system generates a second historical set of patient data (e.g., exemplary data set 800). The second historical set of patient data includes data from a second plurality of patients having one or more phenotypic differences regarding patient features and/or one or more respiratory conditions. In some examples, the phenotypic differences include data regarding one or more respiratory conditions. In some examples, the data regarding one or more respiratory conditions includes a true diagnosis of asthma, COPD, both asthma and COPD, or neither asthma nor COPD. In these examples, a true diagnosis is a diagnosis that has been confirmed by one or more physicians and/or research scientists. In some examples, the second historical set of patient data is a sub-set of the first historical set of patient data that includes data from one or more patients of the first plurality of patients included in the first historical set of patient data that satisfy the set of one or more data-correlation criteria generated at block 910.

At block 914, the computing system generates a first diagnostic model by applying one or more supervised machine learning algorithms to the second historical set of patient data generated at block 912 (e.g., as described above with reference to block 412 of FIG. 4).

At block 916, the computing system generates a second diagnostic model by applying one or more supervised machine learning algorithms to a third historical set of patient data. The third historical set of patient data includes data from a third plurality of patients having one or more phenotypic differences regarding patient features and/or one or more respiratory conditions. In some examples, the phenotypic differences include data regarding one or more respiratory conditions. In some examples, the data regarding one or more respiratory conditions includes a true diagnosis of asthma, COPD, both asthma and COPD, or neither asthma nor COPD. In these examples, a true diagnosis is a diagnosis that has been confirmed by one or more physicians and/or research scientists. In some examples, the third historical set of patient data and the first historical set of patient data are the same historical set of patient data (e.g., exemplary data set 500). In some examples, the second historical set of patient data generated at block 912 is a sub-set of the third historical set of patient data. In these examples, the second historical set of patient data includes data from one or more patients of the third plurality of patients included in the third historical set of patient data that satisfy the set of one or more data-correlation criteria generated at block 910. As will be discussed in greater detail below, the computing system applies the first diagnostic model generated at block 914 and/or the second diagnostic model generated at block 916 to a patient's data to predict an asthma and/or COPD diagnosis for the patient.

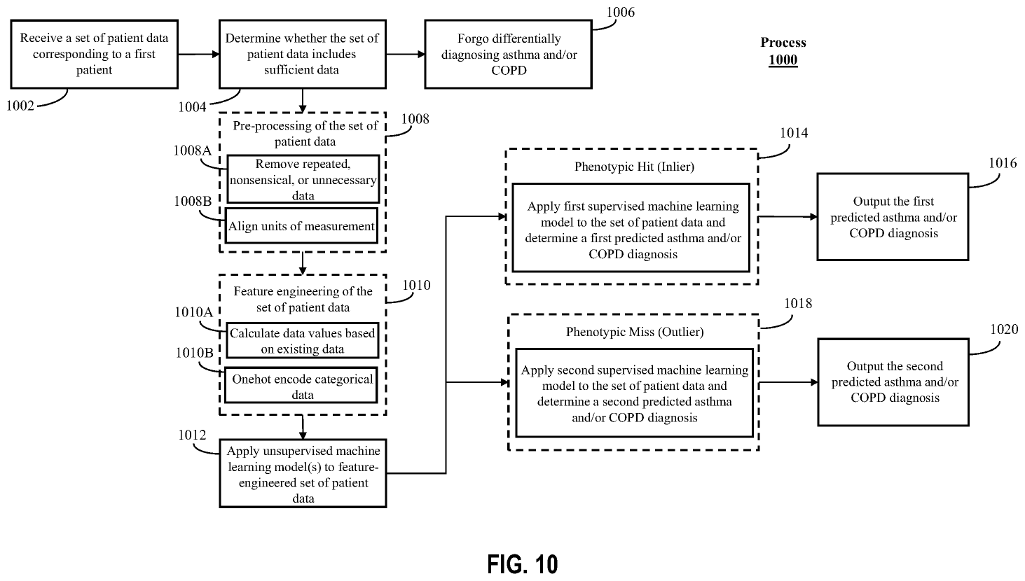

FIG. 10 illustrates an exemplary, computerized process for differentially diagnosing asthma and COPD in a patient. In some examples, process 1000 is performed by a system having one or more features of system 100, shown in FIG. 1. For example, the blocks of process 1000 can be performed by client system 102, cloud computing system 112, and/or cloud computing resource 126.

At block 1002, a computing system (e.g., client system 102, cloud computing system 112, and/or cloud computing resource 126) receives, via one or more input elements (e.g., human input device 312 and/or network interface 310), a set of patient data corresponding to a patient. The set of patient data includes a plurality of data inputs representing the patient's features, physiological measurements, and/or other information relevant to diagnosing asthma and/or COPD. In some examples, the data inputs representing the patient's physiological measurements includes results of at least one physiological test administered to the patient (e.g., a lung function test, an exhaled nitric oxide test (such as a FeNO test), or the like self-administered by the patient, or administered by a physician, clinician, or other individual). Further, in some examples, the computing system receives (e.g., via network interface 310) one or more of the data inputs representing the patient's physiological measurements from one or more physiological test devices over a network (e.g., network 106). Some examples of such physiological test devices include (but are not limited to) a spirometry device, a FeNO device, and a chest radiography (x-ray) device.

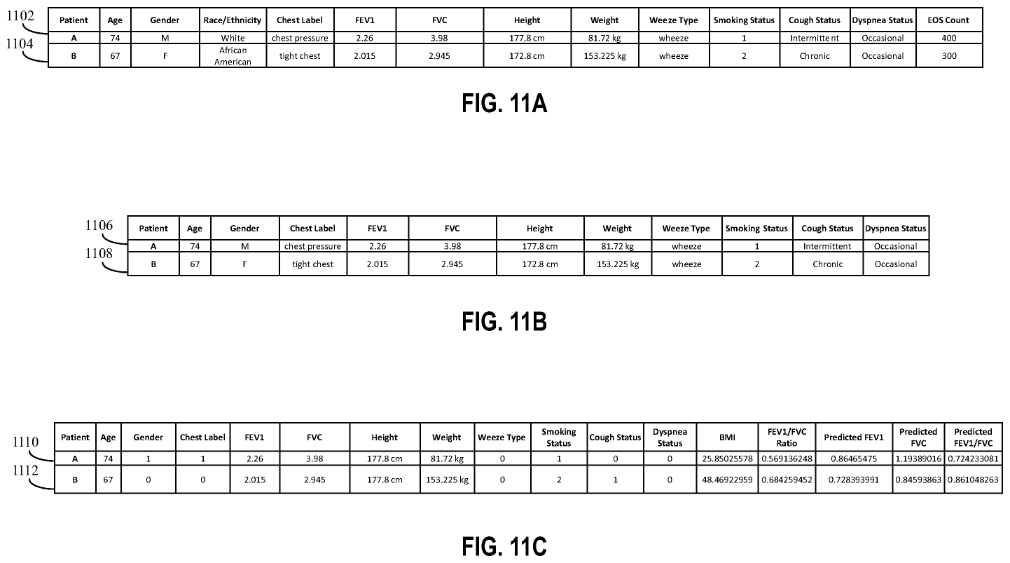

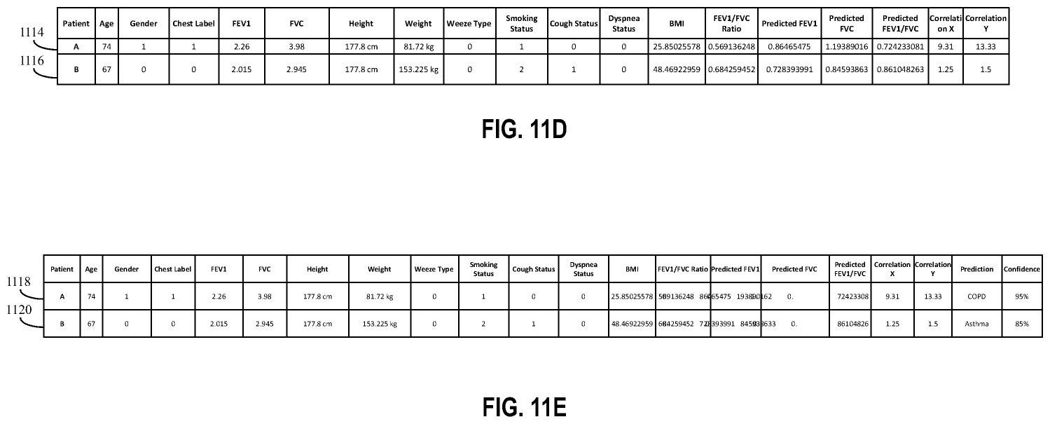

FIG. 11A illustrates two exemplary sets of patient data corresponding to a first patient and a second patient. Specifically, FIG. 11A illustrates exemplary set of patient data 1102 corresponding to Patient A and exemplary set of patient data 1104 corresponding to Patient B. As shown, exemplary set of patient data 1102 and 1104 each include a plurality of data inputs for Patient A and Patient B, respectively. Specifically, the plurality of data inputs include patient age, gender (e.g., male or female), race/ethnicity (e.g., White, Hispanic, Asian, African American, etc.), chest label (e.g., tight chest, chest pressure, etc.), forced expiratory volume in one second (FEV1) measurement, forced vital capacity (FVC) measurement, height, weight, smoking status (e.g., number of cigarette packs per year), cough status (e.g., occasional, intermittent, mild, chronic, etc.), dyspnea status (e.g., exertional, occasional, etc.), and Eosinophil (EOS) count.

In some examples, the set of patient data received at block 1002 includes more data inputs than those shown in exemplary set of patient data 1102 and exemplary set of patient data 1104 of FIG. 11A. Some examples of additional data inputs include (but are not limited to) a patient BMI, FEV1/FVC ratio, median FEV1/FVC ratio (e.g., if a patient's FEV1 and FVC has been measured more than once), wheeze status (e.g., coarse, bilateral, slight, prolonged, etc.), wheeze status change (e.g., increased, decreased, etc.), cough type (e.g., regular cough, productive cough, etc.), dyspnea type (e.g., paroxysmal nocturnal dyspnea, trepopnea, platypnea, etc.), dyspnea status change (e.g., improved, worsened, etc.), chronic rhinitis count (e.g., number of positive diagnoses), allergic rhinitis count (e.g., number of positive diagnoses), gastroesophageal reflux disease count (e.g., number of positive diagnoses), location data (e.g., barometric pressure and average allergen count of patient residence), and sleep data (e.g., average hours of sleep per night). Additionally, in some examples, a set of patient data includes image data. An example of image data includes (but is not limited to) chest radiographs (e.g., x-ray images). In some examples, the set of patient data received at block 1002 includes less data inputs than those shown in exemplary set of patient data 1102 and exemplary set of patient data 1104 of FIG. 11A.

Returning to FIG. 10, at block 1004, the computing system determines whether the set of patient data received at block 1002 includes sufficient data to differentially diagnose asthma and COPD in the patient. Determining whether the set of patient data includes sufficient data includes determining whether the set of patient data satisfies one or more data-sufficiency requirements. In some examples, the one or more data-sufficiency requirements include a requirement that the set of patient data include a minimum number of data inputs. In some examples, the one or more data-sufficiency requirements include a requirement that the set of patient data include one or more core data inputs. Some examples of the one or more core data inputs include (but are not limited to) patient age, gender, height, and/or weight. In some examples, the one or more data-sufficiency requirements include a requirement that one or more data inputs have a specific value range. For example, one such data input value range requirement is a requirement that the patient age data input value be 65 or greater. In some examples, the one or more data-sufficiency requirements are based on the data input values of patients included in the data sets used to generate the first supervised machine learning model and second supervised machine learning model (e.g., as described above with reference to blocks 412 and 414 of FIG. 4). The first supervised machine learning model and the second supervised machine learning model are discussed in greater detail below with respect to block 1014 and block 1018.

At block 1006, in accordance with a determination that the set of patient data received at block 1002 does not include sufficient data, the computing system forgoes differentially diagnosing asthma and COPD in the patient.

At block 1008, in accordance with a determination that the set of patient data received at block 1002 does include sufficient data, the computing device pre-processes the set of patient data. As shown in FIG. 10, pre-processing the set of patient data at block 1008 includes removing repeated, nonsensical, or unnecessary data from the set of patient data at block 1008A and aligning units of measurement for data input values included in the set of patient data at block 1008B. In some examples, removing repeated, nonsensical, or unnecessary data at block 1008A includes removing repeated, nonsensical, and/or unnecessary data inputs from the set of patient data. For example, a data input is unnecessary if the data input has not been identified (e.g., by physicians and research scientists) as being important to the diagnosis of asthma and/or COPD. In some examples, a data input is unnecessary if, based on chi-square and/or ANOVA F-test statistics previously calculated by the computing system (e.g., as described above with reference to block 406 of FIG. 4), the data input is likely to be independent of class and therefore unhelpful for differentially diagnosis asthma and COPD. As shown, pre-processing the set of patient data at block 1008 further includes aligning units of measurement for one or more data input values. In some examples, aligning units of measurement includes converting all data input values to corresponding metric values (where applicable). For example, converting data input value values to corresponding metric values includes converting the value for patient height in the set of patient data to centimeters (cm) and/or converting the value for patient weight in the set of patient data to kilograms (kg).

In some examples, block 1008 does not include one of block 1008A and block 1008B. For example, block 1008 does not include block 808A if there is no repeated, nonsensical, or unnecessary data in the data set received at block 1002. In some examples, block 1008 does not include block 1008B if all of the units of measurement for data input values included in the set of patient data received at block 1002 are already aligned (e.g., already in metric units).

FIG. 11B illustrates two exemplary sets of patient data corresponding to a first patient and a second patient after pre-processing. Specifically, FIG. 11B illustrates exemplary set of patient data 1106 corresponding to Patient A and exemplary set of patient data 1108 corresponding to Patient B, which are generated by the computing system based on the pre-processing of exemplary set of patient data 1102 corresponding to Patient A and exemplary set of patient data 1104 corresponding to Patient B of FIG. 11A. As shown, the computing system removed the race/ethnicity data input from exemplary set of patient data 1102 and exemplary set of patient data 1104. In this example, the computing system removed the patient race/ethnicity data input from exemplary set of patient data 1102 and exemplary set of patient data 1104 based on a determination that patient race/ethnicity is an unnecessary data input. Specifically, the computing system determined that patient race/ethnicity is an unnecessary data input because, in this example, patient race/ethnicity had not been identified (e.g., by physicians and research scientists) as being important to the diagnosis of asthma and/or COPD.

Further, the computing system removed the patient EOS count data input from exemplary set of patient data 1102 and exemplary set of patient data 1104 because based on chi-square statistics previously calculated by the computing system, EOS count is likely to be independent of class and therefore unhelpful for differentially diagnosis asthma and COPD. The pre-processing in this example did not include the computing system aligning units of measurement because the units of measurement of exemplary set of patient data 1102 and exemplary set of patient data 1104 example were already aligned (e.g., patient height data input values were already in cm, patient weight data input values were already in kg, etc.).

Returning to FIG. 10, at block 1010, the computing system feature-engineers the pre-processed set of patient data generated at block 1008. As shown, feature-engineering the pre-processed set of patient data at block 1010 includes calculating (e.g., extrapolating and/or imputing) values for one or more new data inputs based on the values of one or more data inputs of the patient's plurality of data inputs at block 1010A. Some examples of values for the one or more new data inputs that the computing system calculates include (but are not limited to) patient BMI, FEV1/FVC ratio, predicted FEV1, predicted FVC, and/or predicted FEV1/FVC ratio (e.g., a ratio of predicted FEV1 over predicted FVC). In some examples, calculating the values for the one or more new data inputs based on the values of one or more data inputs of the patient's plurality of data inputs includes calculating the values for the one or more new data inputs based on existing models available within relevant research and/or academic literature (e.g., calculating a value for a predicted patient FEV1 data input based on patient gender and race data input values). In some examples, calculating the values for the one or more new data inputs based on the values of one or more data inputs of the patient's plurality of data inputs includes calculating the values for the one or more new data inputs based on patient age, gender, and/or race/ethnicity matched averages (e.g., averages provided by physicians and/or research scientists, averages within relevant research and/or academic literature, etc.). After calculating values for one or more new data inputs, the computing system adds/imputes the one or more new data inputs to the set of patient data.

Feature-engineering the pre-processed set of patient data at block 1010 further includes the computing system onehot encoding categorical data inputs (e.g., data inputs having non-numerical values) included in the set of patient data at block 1010B. Onehot encoding categorical data inputs included in the set of patient data includes converting each of the non-numerical data input values in the set of patient data into numerical values and/or binary values representing the non-numerical data input values. For example, converting non-numerical data input values into binary values includes the computing system converting non-numerical data input values “tight chest” and “chest pressure” for the patient chest label data input into binary values 0 and 1, respectively.

FIG. 11C illustrates two exemplary sets of patient data after feature engineering. Specifically, FIG. 11C illustrates exemplary set of patient data 1110 corresponding to Patient A and exemplary set of patient data 1112 corresponding to Patient B, which are generated by the computing system based on the feature engineering of exemplary set of patient data 1106 and exemplary set of patient data 1108. As shown, the computing system calculated values for five new data inputs for both Patient A and Patient B, and subsequently added the new data inputs to exemplary set of patient data 1106 and exemplary set of patient data 1108. Specifically, the computing system calculated values, and added new data inputs for, patient BMI, FEV1/FVC ratio, predicted FEV1, predicted FVC, and predicted FEV1/FVC ratio for Patient A and Patient B. As explained above, the computing system could have calculated the values for these new data inputs based on (1) the values of one or more data inputs for each patient, (2) existing models available within relevant research and/or academic literature, and/or (3) patient age and/or gender matched averages (but not race/ethnicity matched averages, as the race/ethnicity data inputs were removed during the pre-processing of both exemplary sets of patient data). For example, the computing system could have determined the values for the patient BMI data input based on existing models for calculating BMI and the values of the height and weight data inputs for Patient A and Patient B included in exemplary set of patient data 1106 and exemplary set of patient data 1108, respectively.

As shown in FIG. 11C, the computing system also onehot encoded values of several categorical data inputs for both Patient A and Patient B. Specifically, the computing system converted the non-numerical values for the patient gender, chest label, wheeze type, cough status, and dyspnea status categorical data inputs included in exemplary set of patient data 1106 and exemplary set of patient data 1108 into binary values representing the non-numerical values. For example, with respect to the patient chest label data input, the computing device converted the “tight chest” value for Patient B to a binary value of “0” and the “chest pressure” value for Patient A to a binary value of “1.” As another example, with respect to the wheeze type data input, the computing device converted the “Wheeze” values for both Patient A and Patient B to a binary value of “0.” The computing system made similar conversions for the patient gender, cough status, and dyspnea status data inputs for both Patient A and Patient B.

Returning to FIG. 10, at block 1012, the computing system applies two unsupervised machine learning models to the feature-engineered set of patient data generated at block 1010. First, the computing system applies a UMAP model to the set of patient data. The UMAP model is generated by the computing system's application of a UMAP algorithm to a training data set of patients (e.g., as described above with reference to block 408 of FIG. 4). The computing system's application of the UMAP model to the set of patient data non-linearly reduces the number of dimensions in the set of patient data and generates a reduced-dimension representation of the set of patient data in the same manner that the computing system non-linearly reduced the number of dimensions in the training data set and generated a reduced-dimension representation of the training data set. In some examples, the reduced-dimension representation of the set of patient data includes a reduced-dimension representation of the patient's data input values in the form of one or more coordinates (e.g., in the form of two-dimensional x and y coordinates).

In some examples, after generating a reduced-dimension representation of the patient's data input values (e.g., in the form of one or more coordinates), the computing system adds the reduced-dimension representation to the set of patient data as one or more new data inputs. For example, in the example above wherein the computing system generates a two-dimensional representation of the patient's data input values in the form of two-dimensional coordinates, the computing system subsequently adds a new data input for each coordinate of the two-dimensional coordinates to the set of patient data.

After generating a reduced-dimension representation of the patient's data input values using the UMAP model, the computing system applies an HDBSCAN model to the reduced-dimension representation of the set of patient data (e.g., generated via the application of the UMAP model to the set of patient data). The HDBSCAN model is generated by the computing system's application of an HDBSCAN algorithm to the reduced-dimension representation of the training data set discussed above with respect to the UMAP model (e.g., as described above with reference to block 408 of FIG. 4). In some examples, the computing system's application of the HDBSCAN model to the reduced-dimension representation of the set of patient data clusters the patient into one of the one or more clusters previously generated by the computing system's application of the HDBSCAN algorithm to the training data set of patients based on the reduced-dimension representation of the patient's data input values and one or more threshold similarity/correlation requirements (discussed in greater detail below). If the patient is clustered into one of the one or more previously-generated clusters of patients, the patient is referred to as an “inlier” and/or a “phenotypic hit.”

In some examples, the patient is not clustered into one of the one or more previously-generated clusters of patients. A patient that is not clustered into a cluster of the one or more previously-generated clusters of patients is referred to as an “outlier” and/or a “phenotypic miss.” For example, the computing system will not cluster a patient into a cluster of the one or more previously-generated clusters of patients if the computing system determines (based on the application of the HDBSCAN model to the reduced-dimension representation of the set of patient data) that the reduced-dimension representation of the patient's data input values do not satisfy one or more threshold similarity/correlation requirements.

In some examples, the one or more threshold similarity/correlation requirements include a requirement that each coordinate of the reduced-dimension representation of the patient's data input values (e.g., x, y, and z coordinates for a three-dimensional representation) be within a certain numerical range in order to be clustered into one of the one or more previously-generated clusters of patients. In these examples, the certain numerical range is based on the reduced-dimension representation coordinates of the patients clustered in the one or more previously-generated clusters. In some examples, the one or more threshold similarity/correlation requirements include a requirement that at least one coordinate of the reduced-dimension representation of the patient's data input values be within a certain proximity to a corresponding coordinate of a reduced-dimension representation of the data input values for one or more patients in at least one of the one or more previously-generated clusters of patients. In some examples, the one or more threshold similarity/correlation requirements include a requirement that all coordinates of a reduced-dimension representation of the patient's data input values be within a certain proximity to corresponding coordinates of reduced-dimension representations of a minimum number of patients in at least one of the one or more previously-generated clusters of patients. In some examples, the one or more threshold similarity/correlation requirements include a requirement that all coordinates of a reduced-dimension representation of a patient's data input values be within a certain proximity to a cluster centroid (e.g., a center point of a cluster). In these examples, the computing system determines a cluster centroid for each of the one or more previously-generated clusters that the computing system generates based on the application of the HDBSCAN algorithm to the reduced-dimension representation of the training data set of patients described above.

FIG. 11D illustrates two exemplary sets of patient data after the application of two unsupervised machine learning models to the two exemplary sets of patient data. Specifically, FIG. 11D illustrates exemplary set of patient data 1114 corresponding to Patient A and exemplary set of patient data 1116 corresponding to Patient B, which are generated by the computing system after (1) applying a UMAP model to exemplary set of patient data 1110 corresponding to Patient A and exemplary set of patient data 1112 corresponding to Patient B to generate a two-dimensional representation of the data input values for Patient A in exemplary data set 1110 and the data input values for Patient B in exemplary data set 1112, and (2) adding the two-dimensional representation of the data input values for Patient A and Patient B to exemplary set of patient data 1110 and exemplary set of patient data 1112, respectively, in the form of two new data inputs for each patient (e.g., Correlation X and Correlation Y).

As shown in FIG. 11D, Patient A has a Correlation X value of 9.31 and a Correlation Y value of 13.33 whereas Patient B has a Correlation X value of 1.25 and a Correlation Y value of 1.5. As mentioned above, the computing system applies an HDBSCAN model to the Correlation X and Correlation Y values corresponding to Patient A and Patient B to cluster Patient A and/or Patient B into a cluster of one or more previously-generated clusters of patients based on the Correlation X and Correlation Y values of each patient and one or more threshold similarity/correlation requirements. In this example, the one or more previously-generated clusters of patients are the four clusters of patients discussed above with reference to FIG. 8. Accordingly, based on Patient A's and Patient B's Correlation X and Correlation Y values and the one or more threshold similarity/correlation requirements, the computing system clustered Patient A into the cluster of patients containing Patient 2, Patient 6, and Patient 11 (of FIG. 8), but did not cluster Patient B into any of the four clusters of patients. In other words, the computing system determined that Patient A is an inlier/phenotypic hit and that Patient B is an outlier/phenotypic miss.

Returning to FIG. 10, in some examples, at block 1012, the computing system applies a Gaussian mixture model to the feature-engineered set of patient data instead of the UMAP and HDBSCAN models to classify the patient as an inlier or outlier. The Gaussian mixture model is generated by the computing system's application of a Gaussian mixture model algorithm to a training data set of patients (e.g., as described above with reference to block 408 of FIG. 4). For example, the computing system trains the Gaussian mixture model using the same training data set of patients used to train the UMAP model described above. In some examples, the computing system applies a Gaussian mixture model that was trained based on a stratified training data set of patients (e.g., stratified based on a specific data input included in the training data set of patients (e.g., gender, smoking status, FEV1, FEV1/FVC ratio, BMI, number of symptoms, or weight)). In these example, the Gaussian mixture model that the computing system applies to the patient data depends on the patient data value for the specific data input based on which the training data set of patients was stratified. For example, if a Gaussian mixture model was trained based on a training data set of patients that only included data for female patients (e.g., a training data set of patients stratified based on gender), then the computing system would apply the Gaussian mixture model to a set of patient data if the set of patient data indicated that the patient is a female.

In some examples, the computing system's application of a Gaussian mixture model to the feature-engineered set of patient data groups the patient into a covering manifold previously generated by the computing system's application of the Gaussian mixture model algorithm to the training data set of patients (or a stratified subset of the training data set of patients). If the patient is grouped within the previously-generated covering manifold, the patient is referred to as an “inlier” and/or a “phenotypic hit.” In some examples, the patient is not grouped into the previously-generated covering manifold. A patient that is not grouped into the previously-generated covering manifold is referred to as an “outlier” and/or a “phenotypic miss.”

At block 1014, in accordance with a determination that the patient is an inlier/phenotypic hit, the computing system determines a first predicted asthma and/or COPD diagnosis by applying a first supervised machine learning model to the set of patient data. The first supervised machine learning model is a supervised machine learning model generated by the computing system's application of a supervised machine learning algorithm to a training data set of inlier patients (e.g., as described above with reference to block 412 of FIG. 4). The training data set of inlier patients includes one or more of the data inputs included in the set of patient data for a plurality of patients that the computing system determined were inlier patients based on the application of the UNIAP algorithm and the HDBSCAN algorithm to the training data set of patients discussed above with respect to the computing system's generation of the UMAP model and HDBSCAN model (e.g., with reference to block 812). Determining whether the patient is an inlier/phenotypic hit (e.g., using a UNIAP, HBDSCAN, and/or Gaussian mixture model) prior to applying the first supervised machine learning model to the set of patient data helps to ensure that the computing system only applies the first supervised machine learning model to the set of patient data when the set of patient data provides the computing system with sufficient data to make a highly accurate asthma and/or COPD diagnosis. This in turn allows the computing system to determine asthma and/or COPD diagnoses with very high confidence (as will be discussed below).

At block 1016, the computing system outputs the first predicted asthma and/or COPD diagnosis. For example, the first predicted asthma and/or COPD diagnosis is output by display device 314 of FIG. 3.

At block 1018, in accordance with a determination that the patient is an outlier/phenotypic miss, the computing system determines a second predicted asthma and/or COPD diagnosis by applying a second supervised machine learning model to the set of patient data. The second supervised machine learning model is a supervised machine learning model generated by the computing system's application of a supervised machine learning algorithm to a feature-engineered training data set of patients (e.g., as described above with reference to block 414 of FIG. 4). The feature-engineered training data set of patients includes one or more data inputs included in the set of patient data for a plurality of patients prior to the computing system dividing the feature-engineered training data set into inliers/phenotypic hits and outliers/phenotypic misses (e.g., as described above with reference to FIG. 7).

At block 1020, the computing system outputs the second predicted asthma and/or COPD diagnosis. For example, the first predicted asthma and/or COPD diagnosis is output by display device 314 of FIG. 3.

In some examples, the computing system determines a confidence score corresponding to a predicted asthma and/or COPD diagnosis. For example, the computing system determines a confidence score based on the application of a first supervised machine learning model to a set of patient data (as described above with reference to block 1014). In some examples, the computing system determines a confidence score based on the application of a second supervised machine learning model to a set of patient data (as described above with reference to block 1016). In some examples, the computing system outputs a confidence score with a predicted asthma and/or COPD diagnosis. For example, the computing system outputs a confidence score corresponding to the first predicted asthma and/or COPD diagnosis at block 1016 and/or outputs a confidence score corresponding to the second predicted asthma and/or COPD diagnosis at block 1020.

In some examples, a confidence score represents a predictive probability that a predicted asthma and/or COPD diagnosis is correct (e.g., that the patient truly has the predicted respiratory condition(s)). In some examples, determining the predictive probability includes the computing system determining a logit function (e.g., log-odds) corresponding to the predicted asthma and/or COPD diagnosis and subsequently determining the predictive probability based on an inverse of the logit function (e.g., based on an inverse-logit transformation of the log-odds). This predictive probability determination varies based on the data used to train a supervised machine learning model. For example, a supervised machine learning model trained using similar/correlated data (e.g., the first supervised machine learning model) will generate classifications (e.g., predictions) having higher predictive probabilities than a supervised machine learning model trained with dissimilar/uncorrelated data (e.g., the second supervised machine learning model) due in part to uncertainty and variation introduced into the model by the dissimilar/uncorrelated data. In some examples, the computing system determines the predictive probability based on one or more other logistic regression-based methods.

In some examples, in addition to outputting the confidence scores, the computing system outputs (e.g., displays on a display) a visual breakdown of one or more confidence scores that the computing system outputs (e.g., a visual breakdown for each confidence score). A visual breakdown of a confidence score represents how the computing system generated the confidence score by showing the most impactful data input values with respect to the computing system's determination of a corresponding predicted asthma and/or COPD diagnosis (e.g., showing how those data input values push towards or away from the predicted diagnosis). For example, the visual breakdown can be a bar graph that includes a bar for one or more data input values included in the patient data (e.g., the most impactful data input values), with the length or height of each bar representing the relative importance and/or impact that each data input value had in the determination of the predicted diagnosis (e.g., the longer a data input's bar is, the more impact that data input value had on the predicted diagnosis determination).

FIG. 11E illustrates two exemplary sets of patient data after the application of a separate supervised machine learning model to each of the two exemplary sets of patient data. Specifically, FIG. 11E illustrates exemplary set of patient data 1118 corresponding to Patient A and exemplary set of patient data 1120 corresponding to Patient B, both of which include a predicted asthma and/or COPD diagnosis and a corresponding confidence score. As mentioned above with respect to FIG. 11D, the computing system determined that Patient A is an inlier/phenotypic hit and that Patient B is an outlier/phenotypic miss. Thus, because the computing system determined that Patient A is an inlier/phenotypic hit, the computing system determined a predicted COPD diagnosis for Patient A by applying a first supervised machine learning model to Patient A's data input values included in exemplary set of patient data 1114 (e.g., as described above with reference to block 1014). However, because the computing system determined that Patient B is an outlier/phenotypic miss, the computing system determined a predicted asthma diagnosis for Patient B by applying a second supervised machine learning model to Patient B's data input values included in exemplary set of patient data 1116 (e.g., as described above with reference to block 1016).

Further, as shown in FIG. 11E, the computing system determined a confidence score of 95% corresponding to Patient A's predicted COPD diagnosis and a confidence score of 85% corresponding to Patient B's predicted asthma diagnosis. As mentioned above with respect to block 412 of FIG. 4, a benefit of generating a set of inlier patients (such as exemplary data set 800 of FIG. 8) by applying one or more unsupervised machine learning algorithms to a larger set of patients (such as exemplary data set 700 of FIG. 7) and subsequently generating a supervised machine learning model by applying a supervised machine learning algorithm to the set of inlier patients is that the supervised machine learning model can thereafter make predictions (in this case, predicted asthma and/or COPD diagnoses) with greater accuracy/precision (and thus greater confidence) when applied to a patient having similar/correlated data to that of the patients included in the set of inlier patients (e.g., a patient determined to be an inlier/phenotypic hit at block 1012 of FIG. 10). Thus, in this example, Patient A has a very high confidence score of 95% for at least the reason that the computing system determined that Patient A is an inlier/phenotypic hit and thus determined Patient A's predicted COPD diagnosis by applying the first supervised machine learning model to Patient A's data input values. While Patient B's confidence score of 85% is still quite high, it is not as high as Patient A's confidence score for at least the reason that the computing system determined that Patient B is an outlier/phenotypic miss and thus determined Patient B's predicted asthma diagnosis by applying the second supervised machine learning model to Patient B's data input values.

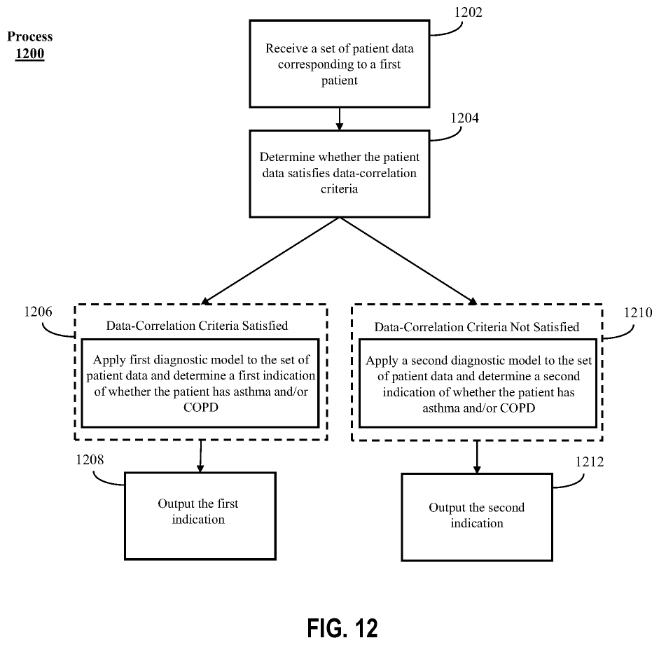

FIG. 12 illustrates an exemplary, computerized process for determining a first indication and a second indication of whether a first patient has one or more respiratory conditions selected from a group consisting of asthma and COPD. In some examples, process 1200 is performed by a system having one or more features of system 100, shown in FIG. 1. For example, the blocks of process 1200 can be performed by client system 102, cloud computing system 112, and/or cloud computing resource 126.

At block 1202, a computing system (e.g., client system 102, cloud computing system 112, and/or cloud computing resource 126) receives a set of patient data corresponding to a first patient (e.g., as described above with reference to block 1002 of FIG. 10). The set of patient data includes a plurality of inputs. In some examples, the plurality of inputs include one or more inputs representing the first patient's age, gender, weight, BMI, and race. In some examples, the set of patient data includes one or more physiological inputs based on the results of one or more physiological tests administered to the first patient using one or more physiological test devices. For example, at least one of the one or more physiological inputs is based on a lung function test administered to the first patient using a spirometry device (e.g., an FEV1 measurement, FVC measurement, FEV1/FVC measurement, etc.) and/or a nitric oxide exhalation test administered to the first patient using a FeNO device (e.g., a nitric oxide measurement). In some examples, the computing system receives the one or more physiological inputs from the one or more physiological test devices over a network (e.g., network 106).

At block 1204, the computing system determines whether the set of patient data corresponding to the first patient satisfies a set of one or more data-correlation criteria (e.g., as described above with reference to block 1012 of FIG. 10). In some examples, the set of one or more data-correlation criteria is based on an application of one or more unsupervised machine learning algorithms (e.g., a UMAP algorithm, HDBSCAN algorithm, and/or Gaussian mixture model algorithm) to a first historical set of patient data (e.g., as described above with reference to block 408 of FIG. 4 and block 910 of FIG. 9). In other examples, the set of one or more data-correlation criteria is based on an application of one or more unsupervised machine learning algorithms (e.g., a Gaussian mixture model algorithm) to one or more stratified subsets of a first historical set of patient data (e.g., stratified based on gender, smoking status, FEV1, FEV1/FVC ratio, BMI, number of symptoms, or weight).

In some examples, the set of one or more data-correlation criteria include one or more unsupervised machine learning models (e.g., one or more unsupervised machine learning model artifacts (e.g., a UMAP model, HDBSCAN model, and/or Gaussian mixture model)) generated by the computing system based on the application of the one or more unsupervised machine learning algorithms to the first historical set of patient data or to a stratified subset of the first historical set of patient data (e.g., as described above with reference to block 408 of FIG. 4 and block 910 of FIG. 9). In these examples, determining whether the set of patient data satisfies the set of one or more data-correlation criteria includes applying the one or more unsupervised machine learning models to the set of patient data and determining, based on the application of the one or more unsupervised machine learning models to the set of patient data, whether the set of patient data is correlated to data corresponding to one or more patients included in the first historical set of patient data (e.g., as described above with reference to block 1012 of FIG. 10).

In some examples, the set of one or more data-correlation criteria includes a requirement that a patient fall within in a cluster of one or more clusters of patients generated by applying the one or more unsupervised machine learning algorithms to the first historical set of patient data (e.g., as described above with reference to block 408 of FIG. 4 and block 910 of FIG. 9). In these examples, determining whether the set of patient data satisfies the set of one or more data-correlation criteria includes determining whether the first patient falls within a cluster of the one or more clusters of patients (e.g., the set of patient data corresponding to the first patient satisfies the set of one or more data-correlation criteria if the patient falls within a cluster of the one or more clusters of patients).

In other examples, the set of one or more data-correlation criteria includes a requirement that a patient fall within a covering manifold of patients generated by applying the one or more unsupervised machine learning algorithms to the feature-engineered first historical set of patient data (or to a stratified subset of the feature-engineered first historical set of patient data (e.g., stratified based on gender, smoking status, FEV1, FEV1/FVC ratio, BMI, number of symptoms, or weight)). In these examples, determining whether the set of patient data satisfies the set of one or more data-correlation criteria includes determining whether the first patient falls within the covering manifold (e.g., the set of patient data corresponding to the first patient satisfies the set of one or more data-correlation criteria if the patient falls within the covering manifold).

At block 1206, in accordance with a determination that the set of patient data corresponding to the first patient satisfies the set of one or more data-correlation criteria, the computing system determines a first indication of whether the first patient has one or more respiratory conditions selected from a group consisting of asthma and COPD based on an application of a first diagnostic model to the set of patient data corresponding to the first patient (e.g., as described above with reference to block 1014 of FIG. 10). The first diagnostic model is based on an application of a first supervised machine learning algorithm to a second historical set of patient data (e.g., as described above with reference to block 412 of FIG. 4 and block 914 of FIG. 9). In some examples, the application of the first supervised machine learning algorithm to the second historical set of patient data occurs at one or more cloud computing systems of the computing system (e.g., cloud computing system 112 and/or cloud computing resource 126). In these examples, a user device of the computing system (e.g., client system 102) receives the first diagnostic model over a network (e.g., network 106) from the one or more cloud computing systems.

At block 1208, the computing system outputs the first indication of whether the first patient has one or more respiratory conditions selected from a group consisting of asthma and COPD (e.g., as described above with reference to block 1016 of FIG. 10).

At block 1210, in accordance with a determination that the set of patient data corresponding to the first patient does not satisfy the set of one or more data-correlation criteria, the computing system determines a second indication of whether the first patient has one or more respiratory conditions selected from a group consisting of asthma and COPD based on an application of a second diagnostic model to the set of patient data corresponding to the first patient (e.g., as described above with reference to block 1018 of FIG. 10). The second diagnostic model is based on an application of a second supervised machine learning algorithm to a third set of patient data (e.g., as described above with reference to block 414 of FIG. 4 and block 916 of FIG. 9). In some examples, the application of the second supervised machine learning algorithm to the third historical set of patient data occurs at one or more cloud computing systems of the computing system (e.g., cloud computing system 112 and/or cloud computing resource 126). In these examples, a user device of the computing system (e.g., client system 102) receives the second diagnostic model over a network (e.g., network 106) from the one or more cloud computing systems.

At block 1212, the computing system outputs the second indication of whether the first patient has one or more respiratory conditions selected from a group consisting of asthma and COPD (e.g., as described above with reference to block 1020 of FIG. 10).

What is claimed is:

1. A system, comprising:
one or more processors;
one or more input elements;
memory; and
one or more programs stored in the memory, the one or more programs including instructions for:
receiving, via the one or more input elements, a set of patient data corresponding to a first patient, the set of patient data including at least one physiological input based on results of at least one physiological test administered to the first patient;
determining, based on the set of patient data, whether a set of one or more data-correlation criteria are satisfied, wherein the set of one or more data-correlation criteria are based on an application of an unsupervised machine learning algorithm to a first historical set of patient data that includes data from a first plurality of patients having one or more phenotypic differences, the phenotypic differences including at least data regarding one or more respiratory conditions;
in accordance with a determination that the set of one or more data-correlation criteria are satisfied:
determining a first indication of whether the first patient has one or more respiratory conditions selected from a group consisting of asthma and chronic obstructive pulmonary disease (COPD) based on an application of a first diagnostic model to the set of patient data, wherein the first diagnostic model is based on an application of a first supervised machine learning algorithm to a second historical set of patient data that includes data from a second plurality of patients having one or more phenotypic differences, the phenotypic differences including at least data regarding one or more respiratory conditions; and
outputting the first indication;
in accordance with a determination that the set of one or more data-correlation criteria are not satisfied:
determining a second indication of whether the first patient has one or more respiratory conditions selected from a group consisting of asthma and chronic obstructive pulmonary disease (COPD) based on an application of a second diagnostic model to the set of patient data,
wherein the second diagnostic model is based on an application of a second supervised machine learning algorithm to a third historical set of patient data that includes data from a third plurality of patients having one or more phenotypic differences, the phenotypic differences including at least data regarding one or more respiratory conditions, and
wherein the third historical set of patient data is different from the second historical set of patient data; and
outputting the second indication.

2. The system of claim 1, wherein the one or more programs further include instructions for determining, based on the application of the first diagnostic model to the set of patient data, a first confidence score corresponding to the first indication.

3. The system of claim 1, wherein the one or more programs further include instructions for determining, based on the application of the second diagnostic model to the set of patient data, a second confidence score corresponding to the second indication.

4. The system of claim 1, wherein the one or more programs further include instructions for determining, based on at least the patient data, whether a set of one or more data-sufficiency criteria are satisfied, and
wherein the determination of whether the set of one or more data-correlation criteria are satisfied is performed in accordance with a determination that the one or more data-sufficiency criteria are satisfied.

5. The system of claim 4, wherein the set of one or more data-sufficiency criteria are satisfied if the set of patient data includes an input indicating that the first patient is over the age of 65.

6. The system of claim 4, wherein the set of one or more data-sufficiency criteria are satisfied if the set of patient data includes at least one of a patient age input, a patient sex input, a patient height input, or a patient weight input.

7. The system of claim 1, wherein the set of patient data includes a plurality of inputs comprising one or more inputs selected from a group consisting of the first patient's age, sex, weight, body mass index, and race.

8. The system of claim 1, wherein the at least one physiological test administered to the patient includes a lung function test administered to the patient using a spirometry device.

9. The system of claim 8, wherein the at least one physiological input is received from the spirometry device.

10. The system of claim 1, wherein the at least one physiological input includes one or more physiological inputs selected from a group consisting of a forced expiratory volume in one second (FEV1) measurement, a forced vital capacity (FVC) measurement, and a ratio of the FEV1 measurement to the FVC measurement (FEV1/FVC ratio).

11. The system of claim 1, wherein the at least one physiological test administered to the patient includes an exhaled nitric oxide test administered to the patient using a fractional exhaled nitric oxide (FeNO) device.

12. The system of claim 1, wherein the application of the of the unsupervised machine learning algorithm to the first historical set of patient data occurs at one or more servers, and wherein the computing device receives the set of one or more data-correlation criteria from the one or more servers.

13. The system of claim 1, wherein the data regarding one or more respiratory conditions included in the first historical set of patient data includes a true diagnosis of asthma, COPD, both asthma and COPD, or neither asthma nor COPD.

14. The system of claim 1, wherein the set of one or more data-correlation criteria includes a requirement that a patient fall within a cluster of one or more clusters of patients generated based on the application of the one or more unsupervised machine learning algorithms to the first historical set of patient data, and
wherein determining, based on the set of patient data, whether the set of one or more data-correlation criteria are satisfied comprises determining, based on the set of patient data, whether the first patient falls within a cluster of the one or more clusters of patients.

15. The system of claim 14, wherein determining, based on the set of patient data, whether the first patient falls within a cluster of the one or more clusters of patients comprises applying one or more unsupervised machine learning models to the set of patient data,
wherein the one or more unsupervised machine learning models are based on the application of the one or more unsupervised machine learning algorithms to the first historical set of patient data.

16. The system of claim 1, wherein the set of one or more data-correlation criteria includes a requirement that a patient fall within a covering manifold generated based on the application of the one or more unsupervised machine learning algorithms to at least a portion of the first historical set of patient data, and
wherein determining, based on the set of patient data, whether the set of one or more data-correlation criteria are satisfied comprises determining, based on the set of patient data, whether the first patient falls within the covering manifold.

17. The system of claim 1, wherein the application of the first supervised machine learning algorithm to the second historical set of patient data occurs at one or more servers, and
wherein the computing device receives the first diagnostic model from the one or more servers.

18. The system of claim 1, wherein the second historical set of patient data is a sub-set of the third historical set of patient data that includes data from one or more patients of the third plurality of patients that satisfies the set of one or more data-correlation criteria.

19. The system of claim 1, wherein the application of the second supervised machine learning algorithm to the third historical set of patient data occurs at one or more servers, and wherein the computing device receives the second diagnostic model from the one or more servers.

20. The system of claim 1, wherein the first supervised machine learning algorithm and the second supervised machine learning algorithm are the same supervised machine learning algorithm.

21. The system of claim 1, wherein the third historical set of patient data and the first historical set of patient data are the same historical set of patient data.

22. The system of claim 1, wherein outputting the indication comprises displaying the indication on a display of the computing device.

23. The system of claim 1, wherein the computing device is a mobile device.

24. The system of claim 1, wherein the computing device is one or more servers.

25. A method, comprising:
at a computing system including one or more processors and one or more input elements:
receiving, via the one or more input elements, a set of patient data corresponding to a first patient, the set of patient data including at least one physiological input based on results of at least one physiological test administered to the first patient;
determining, based on the set of patient data, whether a set of one or more data-correlation criteria are satisfied, wherein the set of one or more data-correlation criteria are based on an application of an unsupervised machine learning algorithm to a first historical set of patient data that includes data from a first plurality of patients having one or more phenotypic differences, the phenotypic differences including at least data regarding one or more respiratory conditions;
in accordance with a determination that the set of one or more data-correlation criteria are satisfied:
determining a first indication of whether the first patient has one or more respiratory conditions selected from a group consisting of asthma and chronic obstructive pulmonary disease (COPD) based on an application of a first diagnostic model to the set of patient data, wherein the first diagnostic model is based on an application of a first supervised machine learning algorithm to a second historical set of patient data that includes data from a second plurality of patients having one or more phenotypic differences, the phenotypic differences including at least data regarding one or more respiratory conditions; and
outputting the first indication;
in accordance with a determination that the set of one or more data-correlation criteria are not satisfied:
determining a second indication of whether the first patient has one or more respiratory conditions selected from a group consisting of asthma and chronic obstructive pulmonary disease (COPD) based on an application of a second diagnostic model to the set of patient data,
wherein the second diagnostic model is based on an application of a second supervised machine learning algorithm to a third historical set of patient data that includes data from a third plurality of patients having one or more phenotypic differences, the phenotypic differences including at least data regarding one or more respiratory conditions, and
wherein the third historical set of patient data is different from the second historical set of patient data; and
outputting the second indication.

26. A non-transitory computer-readable storage medium storing one or more programs configured to be executed by one or more processors of an electronic device with one or more input elements, the one or more programs including instructions for:
receiving, via the one or more input elements, a set of patient data corresponding to a first patient, the set of patient data including at least one physiological input based on results of at least one physiological test administered to the first patient;
determining, based on the set of patient data, whether a set of one or more data-correlation criteria are satisfied, wherein the set of one or more data-correlation criteria are based on an application of an unsupervised machine learning algorithm to a first historical set of patient data that includes data from a first plurality of patients having one or more phenotypic differences, the phenotypic differences including at least data regarding one or more respiratory conditions;
in accordance with a determination that the set of one or more data-correlation criteria are satisfied:
determining a first indication of whether the first patient has one or more respiratory conditions selected from a group consisting of asthma and chronic obstructive pulmonary disease (COPD) based on an application of a first diagnostic model to the set of patient data, wherein the first diagnostic model is based on an application of a first supervised machine learning algorithm to a second historical set of patient data that includes data from a second plurality of patients having one or more phenotypic differences, the phenotypic differences including at least data regarding one or more respiratory conditions; and
outputting the first indication;
in accordance with a determination that the set of one or more data-correlation criteria are not satisfied:
determining a second indication of whether the first patient has one or more respiratory conditions selected from a group consisting of asthma and chronic obstructive pulmonary disease (COPD) based on an application of a second diagnostic model to the set of patient data,
wherein the second diagnostic model is based on an application of a second supervised machine learning algorithm to a third historical set of patient data that includes data from a third plurality of patients having one or more phenotypic differences, the phenotypic differences including at least data regarding one or more respiratory conditions, and
wherein the third historical set of patient data is different from the second historical set of patient data; and
outputting the second indication.
